(19) United States
(12) Patent Application Publication
(10) Pub. No.: US 2018/0071010 A1
(43) Pub. Date: Mar. 15, 2018

(54) TEMPERATURE MONITORING ELECTROSURGICAL SYSTEM

(71) Applicant: Kogent Surgical, LLC, Chesterfield, MO (US)

(72) Inventors: Gregg D. Scheller, Wildwood, MO (US); William J. Bowers, Westminster, CO (US)

(73) Assignee: Kogent Surgical, LLC, Chesterfield, MO (US)

(21) Appl. No.: 15/676,258

(22) Filed: Aug. 14, 2017

Related U.S. Application Data
(60) Provisional application No. 62/393,351, filed on Sep. 12, 2016.

(51) Int. Cl. A61B 18/12; A61B 18/14

(52) U.S. Cl. CPC A61B 18/1206; A61B 2018/00595; A61B 18/1445

(57) ABSTRACT

A temperature monitoring electrosurgical system may include a first forceps arm, a second forceps arm, a temperature monitoring system, and a system control. The first forceps arm may include a first thermocouple electrically connected to a first temperature sense of the temperature monitoring system. The second forceps arm may include a second thermocouple electrically connected to a second temperature sense of the temperature monitoring system. The system control may be configured to compare a measured temperature of the first thermocouple and the second thermocouple to a user defined cauterization temperature setting. The system control may be configured to increase a bipolar output power if the measured temperature is less than the cauterization temperature setting. The system control may be configured to decrease the bipolar output power if the measured temperature is greater than the cauterization temperature setting.

### CROSS-REFERENCE TO RELATED APPLICATIONS

This Application claims the benefit of U.S. Provisional Application No. 62/393,351, filed Sep. 12, 2016.

### FIELD OF THE INVENTION

The present disclosure relates to a medical device, and, more particularly, to an electrosurgical system.

### BACKGROUND OF THE INVENTION

A variety of complete surgical procedures and portions of surgical procedures may be performed with bipolar forceps, e.g., bipolar forceps are commonly used in dermatological, gynecological, cardiac, plastic, ocular, spinal, maxillofacial, orthopedic, urological, and general surgical procedures. Bipolar forceps are also used in neurosurgical procedures; however, the use of bipolar forceps in neurosurgical procedures presents unique risks to patients if the surgeon is unable to both visually and tactilely confirm that an electrosurgical procedure is being performed as intended. When a bipolar forceps is used to cauterize a target tissue it is important that no more thermal energy is applied to the target tissue than necessary to achieve cauterization. Any excess thermal energy applied to the target tissue may spread to a non-target tissue or cause the target tissue to stick to a portion of the bipolar forceps.

### BRIEF SUMMARY OF THE INVENTION

A temperature monitoring electrosurgical system is presented. In one or more embodiments, a temperature monitoring electrosurgical system may comprise a first forceps arm, a second forceps arm, a temperature monitoring system, and a system control. Illustratively, the first forceps arm may comprise a first thermocouple electrically connected to a first temperature sense of the temperature monitoring system. In one or more embodiments, the second forceps arm may comprise a second thermocouple electrically connected to a second temperature sense of the temperature monitoring system. Illustratively, the system control may be configured to compare a measured temperature of the first thermocouple and the second thermocouple to a user defined cauterization temperature setting. In one or more embodiments, the system control may be configured to increase a bipolar output power if the measured temperature is less than the cauterization temperature setting. Illustratively, the system control may be configured to decrease the bipolar output power if the measured temperature is greater than the cauterization temperature setting.

### BRIEF DESCRIPTION OF THE DRAWINGS

The above and further advantages of the present invention may be better understood by referring to the following description in conjunction with the accompanying drawings in which like reference numerals indicate identical or functionally similar elements:

FIGS. 1A and 1B are schematic diagrams illustrating a forceps arm;

FIG. 2 is a schematic diagram illustrating an exploded view of a temperature monitoring bipolar forceps assembly;

FIG. 3 is a schematic diagram illustrating an assembled temperature monitoring bipolar forceps;

FIGS. 4A, 4B, and 4C are schematic diagrams illustrating a gradual closing of a temperature monitoring bipolar forceps;

FIGS. 5A, 5B, and 5C are schematic diagrams illustrating a uniform compression of a vessel;

FIG. 6 is a schematic block diagram illustrating a temperature monitoring electrosurgical system;

FIG. 7 is a schematic block diagram illustrating a power supply;

FIG. 8 is a schematic block diagram illustrating a system control;

FIG. 9 is a schematic block diagram illustrating a temperature monitoring system;

FIG. 10 is a schematic block diagram illustrating an RF system;

FIG. 11 is a flowchart illustrating a tissue cauterization;

FIG. 12 is a flowchart illustrating a system activation;

FIG. 13 is a flowchart illustrating an establishment of tissue cauterization parameters;

FIG. 14 is a flowchart illustrating a monitoring and adjustment of a tissue cauterization.

### DETAILED DESCRIPTION OF AN ILLUSTRATIVE EMBODIMENT

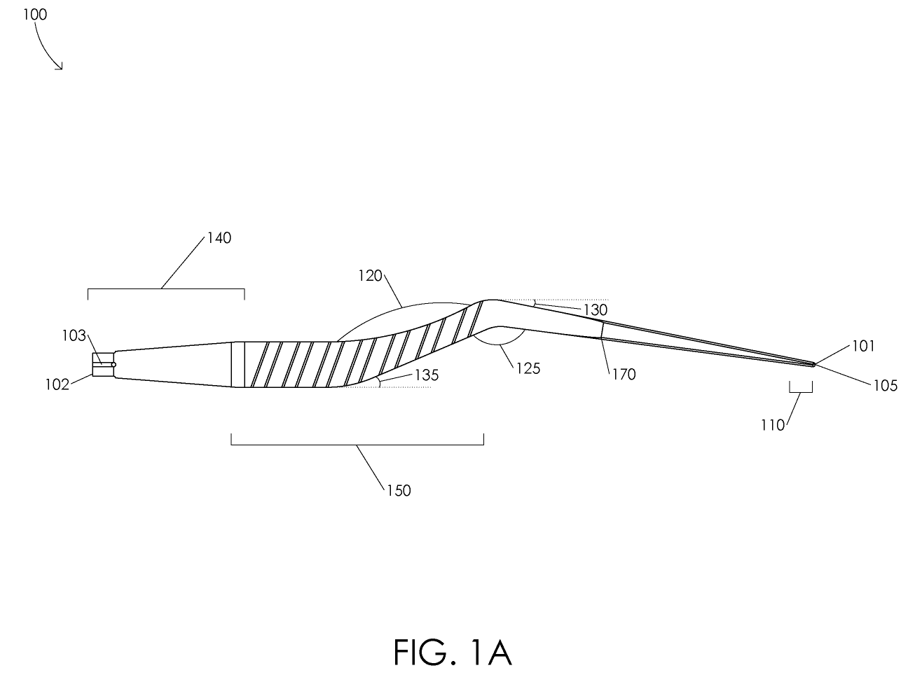

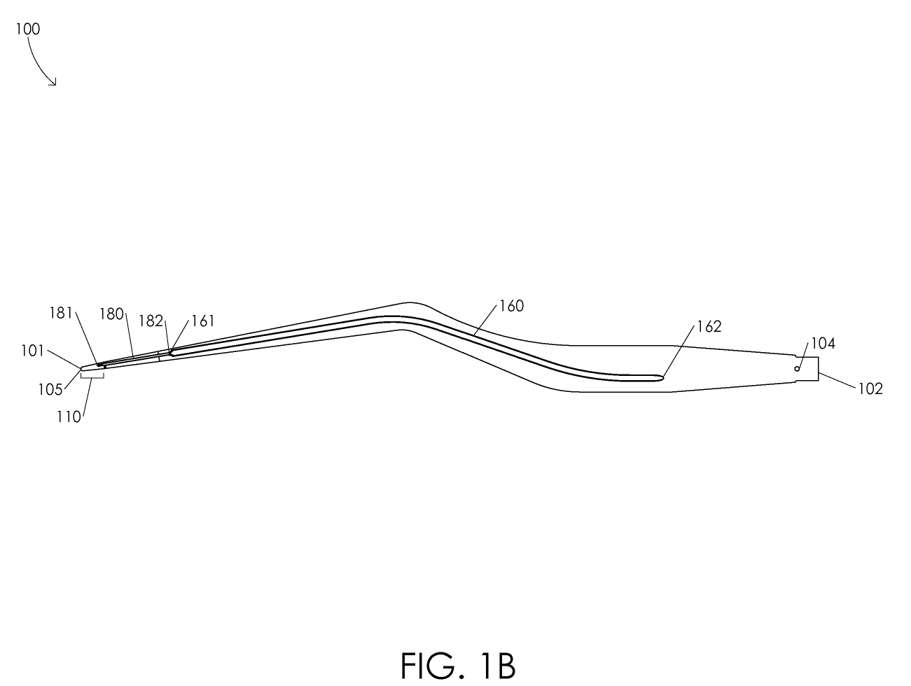

FIGS. 1A and 1B are schematic diagrams illustrating a forceps arm 100. FIG. 1A is a schematic diagram illustrating a lateral view of a forceps arm 100. Illustratively, a forceps arm 100 may comprise an input conductor housing 103, a thermocouple housing 105, a conductor tip 110, a forceps arm superior incline angle 120, a forceps arm inferior decline angle 125, a forceps arm superior decline angle 130, a forceps arm inferior incline angle 135, a spring 140, a forceps arm grip 150, and a forceps jaw taper interface 170. FIG. 1B is a schematic diagram illustrating a medial view of a forceps arm 100. Illustratively, a forceps arm 100 may comprise a proximal channel 160 having a proximal channel distal end 161 and a proximal channel proximal end 162 and a distal channel 180 having a distal channel distal end 181 and a distal channel proximal end 182. In one or more embodiments, forceps arm 100 may be may be manufactured from any suitable material, e.g., polymers, metals, metal alloys, etc., or from any combination of suitable materials. Illustratively, forceps arm 100 may be manufactured from an electrically conductive material, e.g., metal, graphite, conductive polymers, etc. In one or more embodiments, forceps arm 100 may be manufactured from an electrically conductive metal, e.g., silver, copper, gold, aluminum, etc. Illustratively, forceps arm 100 may be manufactured from an electrically conductive metal alloy, e.g., a silver alloy, a copper alloy, a gold alloy, an aluminum alloy, stainless steel, etc.

In one or more embodiments, forceps arm 100 may be manufactured from a material having an electrical conductivity in a range of 30.0×10⁶ to 40.0×10⁶ Siemens per meter at a temperature of 20.0° C., e.g., forceps arm 100 may be manufactured from a material having an electrical conductivity of 35.5×10⁶ Siemens per meter at a temperature of 20.0° C. Illustratively, forceps arm 100 may be manufactured from a material having an electrical conductivity of less than 30.0×10⁶ Siemens per meter or greater than 40.0×10⁶ Siemens per meter at a temperature of 20.0° C. In one or more embodiments, forceps arm 100 may be manufactured from a material having a thermal conductivity in a range of 180.0 to 250.0 Watts per meter Kelvin at a temperature of 20.0° C., e.g., forceps arm 100 may be manufactured from a material having a thermal conductivity of 204.0 Watts per meter Kelvin at a temperature of 20.0° C. Illustratively, forceps arm 100 may be manufactured from a material having a thermal conductivity of less than 180.0 Watts per meter Kelvin or greater than 250.0 Watts per meter Kelvin at a temperature of 20.0° C. In one or more embodiments, forceps arm 100 may be manufactured from a material having an electrical conductivity in a range of 30.0×10⁶ to 40.0×10⁶ Siemens per meter and a thermal conductivity in a range of 180.0 to 250.0 Watts per meter Kelvin at a temperature of 20.0° C., e.g., forceps arm 100 may be manufactured from a material having an electrical conductivity of 35.5×10⁶ Siemens per meter and a thermal conductivity of 204.0 Watts per meter Kelvin at a temperature of 20.0° C.

Illustratively, forceps arm 100 may have a density in a range of 0.025 to 0.045 pounds per cubic inch, e.g., forceps arm 100 may have a density of 0.036 pounds per cubic inch. In one or more embodiments, forceps arm 100 may have a density less than 0.025 pounds per cubic inch or greater than 0.045 pounds per cubic inch. For example, forceps arm 100 may have a density of 0.0975 pounds per cubic inch. Illustratively, forceps arm 100 may have a mass in a range of 0.0070 to 0.0092 pounds, e.g., forceps arm 100 may have a mass of 0.0082 pounds. In one or more embodiments, forceps arm 100 may have a mass less than 0.0070 pounds or greater than 0.0092 pounds. Illustratively, forceps arm 100 may have a volume in a range of 0.20 to 0.26 cubic inches, e.g., forceps arm 100 may have a volume of 0.227 cubic inches. In one or more embodiments, forceps arm 100 may have a volume less than 0.20 cubic inches or greater than 0.26 cubic inches. Illustratively, forceps arm 100 may have a surface area in a range of 5.0 to 8.0 square inches, e.g., forceps arm 100 may have a surface area of 6.9 square inches. In one or more embodiments, forceps arm 100 may have a surface area less than 5.0 square inches or greater than 8.0 square inches. Illustratively, conductor tip 110 may have a surface area in a range of 0.03 to 0.07 square inches, e.g., conductor tip 110 may have a surface area of 0.053 square inches. In one or more embodiments, conductor tip 110 may have a surface area less than 0.03 square inches or greater than 0.07 square inches. Illustratively, a ratio of forceps arm 100 surface area to conductor tip 110 surface area may be in a range of 100.0 to 180.0, e.g., a ratio of forceps arm 100 surface area to conductor tip 110 surface area may be 137.9. In one or more embodiments, a ratio of forceps arm 100 surface area to conductor tip 110 surface area may be less than 100.0 or greater than 180.0.

Illustratively, conductor tip 110 may be configured to prevent tissue from sticking to conductor tip 110. In one or more embodiments, conductor tip 110 may comprise an evenly polished material configured to prevent tissue sticking. In one or more embodiments, a surface of conductor tip 110 may have a roughness average in a range of 25.0 to 150.0 nanometers, e.g., a surface of conductor tip 110 may have a roughness average of 98.8 nanometers. Illustratively, a surface of conductor tip 110 may have a roughness average of less than 25.0 nanometers or greater than 150.0 nanometers. In one or more embodiments, a surface of conductor tip 110 may have a root mean square average between height deviations over a total surface area of conductor tip 110 in a range of 30.0 to 150.0 nanometers, e.g., a surface of conductor tip 110 may have a root mean square average between height deviations over a total surface area of conductor tip 110 of 112.0 nanometers. Illustratively, a surface of conductor tip 110 may have a root mean square average between height deviations over a total surface area of conductor tip 110 of less than 30.0 nanometers or greater than 150.0 nanometers. In one or more embodiments, a surface of conductor tip 110 may have an average maximum profile of the ten greatest peak-to-valley separations over a total surface area of conductor tip 110 in a range of 100.0 to 850.0 nanometers, e.g., a surface of conductor tip 110 may have an average maximum profile of the ten greatest peak-to-valley separations over a total surface area of conductor tip 110 of 435.0 nanometers. Illustratively, a surface of conductor tip 110 may have an average maximum profile of the ten greatest peak-to-valley separations over a total surface area of conductor tip 110 of less than 100.0 nanometers or greater than 850.0 nanometers. In one or more embodiments, a surface of conductor tip 110 may have a maximum height difference between a highest point and a lowest point of a total surface area of conductor tip 110 in a range of 200.0 to 1300.0 nanometers, e.g., a surface of conductor tip 110 may have a maximum height difference between a highest point and a lowest point of a total surface area of conductor tip 110 of 650.0 nanometers. Illustratively, a surface of conductor tip 110 may have a maximum height difference between a highest point and a lowest point of a total surface area of conductor tip 110 of less than 200.0 nanometers or greater than 1300.0 nanometers.

Illustratively, conductor tip 110 may have a length in a range of 0.22 to 0.3 inches, e.g., conductor tip 110 may have a length of 0.26 inches. In one or more embodiments, conductor tip 110 may have a length less than 0.22 inches or greater than 0.3 inches. Illustratively, conductor tip 110 may have a width in a range of 0.018 to 0.062 inches, e.g., conductor tip 110 may have a width of 0.04 inches. In one or more embodiments, conductor tip 110 may have a width less than 0.018 inches or greater than 0.062 inches. Illustratively, a geometry of forceps arm 100 may comprise a tapered portion, e.g., a tapered portion from forceps jaw taper interface 170 to forceps arm distal end 101. In one or more embodiments, forceps arm 100 may comprise a tapered portion having a tapered angle in a range of 3.0 to 4.5 degrees, e.g., forceps arm 100 may comprise a tapered portion having a tapered angle of 3.72 degrees. Illustratively, forceps arm 100 may comprise a tapered portion having a tapered angle of less than 3.0 degrees or greater than 4.5 degrees.

In one or more embodiments, proximal channel 160 may have a diameter in a range of 1.25 to 1.75 millimeters, e.g., proximal channel 160 may have a diameter of 1.5875 millimeters. Illustratively, proximal channel 160 may have a diameter of less than 1.25 millimeters or greater than 1.75 millimeters. In one or more embodiments, proximal channel 160 may have a depth in a range of 0.035 to 0.040 inches, e.g., proximal channel 160 may have a depth of 0.038 inches. Illustratively, proximal channel 160 may have a depth of less than 0.035 inches or greater than 0.040 inches. In one or more embodiments, proximal channel 160 may have a surface area in a range of 0.5 to 0.8 square inches, e.g., proximal channel 160 may have a surface area of 0.636 square inches. Illustratively, proximal channel 160 may have a surface area of less than 0.5 square inches or greater than 0.8 square inches. In one or more embodiments, proximal channel 160 may have a volume in a range of 0.015 to 0.025 cubic inches, e.g., proximal channel 160 may have a volume of 0.01995 cubic inches. Illustratively, proximal channel 160 may have a volume of less than 0.015 cubic inches or greater than 0.025 cubic inches.

In one or more embodiments, distal channel 180 may have a diameter in a range of 0.75 to 1.25 millimeters, e.g., distal channel 180 may have a diameter of 1.0 millimeters. Illustratively, distal channel 180 may have a diameter of less than 0.75 millimeters or greater than 1.25 millimeters. In one or more embodiments, distal channel 180 may have a depth in a range of 0.035 to 0.040 inches, e.g., distal channel 180 may have a depth of 0.038 inches. Illustratively, distal channel 180 may have a depth of less than 0.035 inches or greater than 0.040 inches. In one or more embodiments, distal channel 180 may have a surface area in a range of 0.04 to 0.08 square inches, e.g., distal channel 180 may have a surface area of 0.066 square inches. Illustratively, distal channel 180 may have a surface area of less than 0.04 square inches or greater than 0.08 square inches. In one or more embodiments, distal channel 180 may have a volume in a range of 0.0005 to 0.0015 cubic inches, e.g., distal channel 180 may have a volume of 0.001 cubic inches. Illustratively, distal channel 180 may have a volume of less than 0.0005 cubic inches or greater than 0.0015 cubic inches. In one or more embodiments, a portion of distal channel 180 may extend a distance into conductor tip 110, e.g., distal channel distal end 181 may be disposed within conductor tip 110. Illustratively, a portion of distal channel 180 may extend a distance in a range of 0.001 to 0.135 inches into conductor tip 110, e.g., distal channel distal end 181 may extend a distance of 0.1 inches into conductor tip 110. In one or more embodiments, a portion of distal channel 180 may extend into conductor tip 110 a distance of less than 0.001 inches or greater than 0.135 inches. Illustratively, a portion of distal channel 180 extending into conductor tip 110 may have a surface area in a range of 0.001 to 0.005 square inches, e.g., a portion of distal channel 180 extending into conductor tip 110 may have a surface area of 0.0033 square inches. In one or more embodiments, a portion of distal channel 180 extending into conductor tip 110 may have a surface area of less than 0.001 square inches or greater than 0.005 square inches.

Illustratively, thermocouple housing 105 may extend from forceps arm distal end 101 to distal channel 180, e.g., thermocouple housing 105 may extend from forceps arm distal end 101 to distal channel distal end 181. In one or more embodiments, thermocouple housing 105 may extend an entire length of conductor tip 110, e.g., thermocouple housing 105 may extend from a distal end of conductor tip 110 to a proximal end of conductor tip 110. Illustratively, thermocouple housing 105 may comprise a bore extending from forceps arm distal end 101, through conductor tip 110, and into distal channel 180. In one or more embodiments, thermocouple housing 105 may be manufactured by drilling a bore into conductor tip 110, e.g., thermocouple housing 105 may be manufactured by drilling a bore into conductor tip 110 extending from forceps arm distal end 101 to distal channel distal end 181. Illustratively, thermocouple housing 105 may have a diameter in a range of 0.090 to 0.015 inches, e.g., thermocouple housing 105 may have a diameter of 0.012 inches. In one or more embodiments, thermocouple housing 105 may have a diameter of less than 0.090 inches or greater than 0.015 inches.

Illustratively, forceps arm 100 may comprise a material having a modulus of elasticity in a range of 9.0×10⁶ to 11.0×10⁶ pounds per square inch, e.g., forceps arm 100 may comprise a material having a modulus of elasticity of 10.0×10⁶ pounds per square inch. In one or more embodiments, forceps arm 100 may comprise a material having a modulus of elasticity less than 9.0×10⁶ pounds per square inch or greater than 11.0×10⁶ pounds per square inch. Illustratively, forceps arm 100 may comprise a material having a shear modulus in a range of 3.5×10⁶ to 4.5×10⁶ pounds per square inch, e.g., forceps arm 100 may comprise a material having a shear modulus of 3.77×10⁶ pounds per square inch. In one or more embodiments, forceps arm 100 may comprise a material having a shear modulus less than 3.5×10⁶ pounds per square inch or greater than 4.5×10⁶ pounds per square inch.

Illustratively, forceps arm superior incline angle 120 may comprise any angle greater than 90.0 degrees. In one or more embodiments, forceps arm superior incline angle 120 may comprise any angle in a range of 150.0 to 170.0 degrees, e.g., forceps arm superior incline angle 120 may comprise a 160.31 degree angle. Illustratively, forceps arm superior incline angle 120 may comprise an angle less than 150.0 degrees or greater than 170.0 degrees. In one or more embodiments, forceps arm inferior decline angle 125 may comprise any angle greater than 90.0 degrees. Illustratively, forceps arm inferior decline angle 125 may comprise any angle in a range of 140.0 to 160.0 degrees, e.g., forceps arm inferior decline angle 125 may comprise a 149.56 degree angle. In one or more embodiments, forceps arm inferior decline angle 125 may comprise an angle less than 140.0 degrees or greater than 160.0 degrees. Illustratively, forceps arm inferior decline angle 125 may comprise any angle less than forceps arm superior incline angle 120, e.g., forceps arm inferior decline angle 125 may comprise an angle in a range of 5.0 to 15.0 degrees less than forceps arm superior incline angle 120. In one or more embodiments, forceps arm inferior decline angle 125 may comprise an angle less than 5.0 degrees or greater than 15.0 degrees less than forceps arm superior incline angle 120.

Illustratively, forceps arm superior decline angle 130 may comprise any angle less than 90.0 degrees. In one or more embodiments, forceps arm superior decline angle 130 may comprise any angle in a range of 5.0 to 15.0 degrees, e.g., forceps arm superior decline angle 130 may comprise an 11.3 degree angle. Illustratively, forceps arm superior decline angle 130 may comprise an angle less than 5.0 degrees or greater than 15.0 degrees. In one or more embodiments, forceps arm inferior incline angle 135 may comprise any angle less than 90.0 degrees. Illustratively, forceps arm inferior incline angle 135 may comprise any angle in a range of 15.0 to 30.0 degrees, e.g., forceps arm inferior incline angle 135 may comprise a 23.08 degree angle. In one or more embodiments, forceps arm inferior incline angle 135 may comprise an angle less than 15.0 degrees or greater than 30.0 degrees. Illustratively, forceps arm inferior incline angle 135 may comprise any angle greater than forceps arm superior decline angle 130, e.g., forceps arm inferior incline angle 135 may comprise an angle in a range of 5.0 to 15.0 degrees greater than forceps arm superior decline angle 130. In one or more embodiments, forceps arm inferior incline angle 135 may comprise an angle less than 5.0 degrees or greater than 15.0 degrees greater than forceps arm superior decline angle 130.

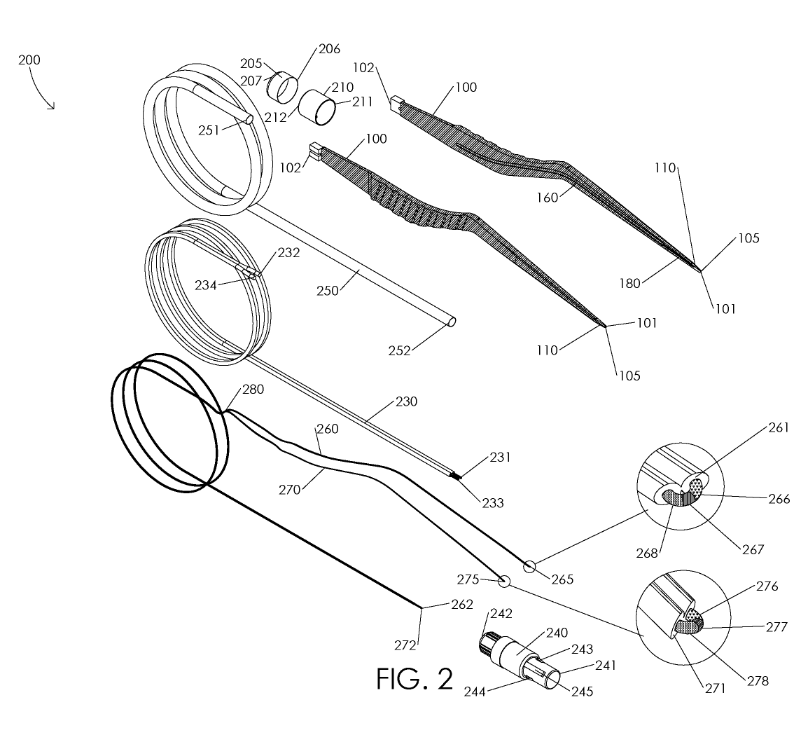

FIG. 2 is a schematic diagram illustrating an exploded view of a temperature monitoring bipolar forceps assembly 200. Illustratively, a temperature monitoring bipolar forceps assembly 200 may comprise a first forceps arm 100, a second forceps arm 100, a housing sleeve interface 205, an input conductor isolation mechanism 210, a bipolar cord 230, a machine connector 240, a housing sleeve 250, a first thermocouple wire 260, and a second thermocouple wire 270. In one or more embodiments, housing sleeve interface 205 may comprise a housing sleeve interface distal end 206 and a housing sleeve proximal end 207. Illustratively, input conductor isolation mechanism 210 may comprise an input conductor isolation mechanism distal end 211 and an input conductor isolation mechanism proximal end 212. In one or more embodiments, housing sleeve interface 205 may be configured to interface with input conductor isolation mechanism 210, e.g., housing sleeve interface distal end 206 may be configured to interface with ins put conductor isolation mechanism proximal end 212. Illustratively, bipolar cord 230 may comprise a first input conductor having a first input conductor and a second input conductor, e.g., the first input conductor may comprise a first input conductor proximal end 231 and a first input conductor distal end 232 and the second input conductor may comprise a second input conductor proximal end 233 and a second input conductor distal end 234.

In one or more embodiments, machine connector 240 may comprise a machine connector distal end 241 and a machine connector proximal end 242. Illustratively, machine connector 240 may comprise a first barb 243, a second barb 244, and a machine connector orientation guide 245. In one or more embodiments, first barb 243 and second barb 244 may be configured to secure an interface between machine connector 240 and an electrosurgical generator. Illustratively, machine connector orientation guide 245 may be configured to ensure that machine connector 240 may only have a single rotational orientation when connected to an electrosurgical generator, e.g., an electrosurgical generator may have an input geometry configured to prevent machine connector 240 from connecting to the electrosurgical generator unless machine connector orientation guide 245 is in a particular orientation relative to the electrosurgical generator input geometry.

In one or more embodiments, housing sleeve 250 may comprise a housing sleeve distal end 251 and a housing sleeve proximal end 252. Illustratively, first thermocouple wire 260 may comprise a first thermocouple wire distal end 261 and a first thermocouple wire proximal end 262. In one or more embodiments, first thermocouple wire 260 may comprise a first thermocouple 265. Illustratively, first thermocouple 265 may be configured to measure a first temperature, e.g., first thermocouple 265 may be configured to measure a first temperature relative to a first reference temperature by measuring a first temperature dependent voltage. In one or more embodiments, first thermocouple 265 may comprise a first wire conductor 266, a second wire conductor 268, and a first thermocouple measuring junction 267. Illustratively, first thermocouple 265 may comprise a Type K thermocouple, e.g., first wire conductor 266 may be manufactured from chromel and second wire conductor 268 may be manufactured from alumel. In one or more embodiments, first thermocouple 265 may comprise a Type E thermocouple, e.g., first wire conductor 266 may be manufactured from chromel and second wire conductor 268 may be manufactured from constantan. Illustratively, first thermocouple 265 may be manufactured from materials that are non-magnetic. In one or more embodiments, first thermocouple 265 may comprise a noble metal thermocouple.

Illustratively, second thermocouple wire 270 may comprise a second thermocouple wire distal end 271 and a second thermocouple wire proximal end 272. In one or more embodiments, second thermocouple wire 270 may comprise a second thermocouple 275. Illustratively, second thermocouple 275 may be configured to measure a second temperature, e.g., second thermocouple 275 may be configured to measure a second temperature relative to a second reference temperature by measuring a second temperature dependent voltage. In one or more embodiments, second thermocouple 275 may comprise a third wire conductor 276, a fourth wire conductor 278, and a second thermocouple measuring junction 277. Illustratively, second thermocouple 275 may comprise a Type K thermocouple, e.g., third wire conductor 276 may be manufactured from chromel and fourth wire conductor 278 may be manufactured from alumel. In one or more embodiments, second thermocouple 275 may comprise a Type E thermocouple, e.g., third wire conductor 276 may be manufactured from chromel and fourth wire conductor 278 may be manufactured from constantan. Illustratively, second thermocouple 275 may be manufactured from materials that are non-magnetic. In one or more embodiments, second thermocouple 275 may comprise a noble metal thermocouple. Illustratively, first thermocouple wire 260 may be configured to interface with second thermocouple wire 270 at a thermocouple wire mechanical union 280.

In one or more embodiments, a portion of each forceps arm 100 may be anodized to increase a thickness of a natural oxide layer on a surface of each forceps arm 100, e.g., a portion of each forceps arm 100 may be coated with an oxide layer greater than 15.0 nanometers. Illustratively, each forceps arm 100 may be subjected to an electrolytic passivation process configured to increase a thickness of an oxide layer on the surface of each forceps arm 100, e.g., a portion of each forceps arm may be coated with an oxide layer greater than 15.0 nanometers. In one or more embodiments, a portion of each forceps arm 100 may be coated with an oxide layer having a thickness in a range of 15.0 nanometers to 1.0 micrometers. Illustratively, a portion of each forceps arm 100 may be coated with an oxide layer having a thickness less than 15.0 nanometers or greater than 1.0 micrometers. In one or more embodiments, an oxide layer on a surface of each forceps arm 100 may be the only electrical insulator material on the surface of each forceps arm 100, e.g., an oxide layer may be the only electrical insulation on a portion of each forceps arm 100.

Illustratively, a portion of each forceps arm 100 may be coated with a thermoplastic material, e.g., a portion of each forceps arm 100 may be coated with nylon. In one or more embodiments, a portion of each forceps arm 100 may be coated with a fluoropolymer, e.g., a portion of each forceps arm 100 may be coated with polyvinylidene fluoride. Illustratively, a portion of each forceps arm 100 may be coated with a material having an electrical conductivity less than 1.0×10⁻⁸ Siemens per meter at a temperature of 20.0° C., e.g., a portion of each forceps arm 100 may be coated with a material having an electrical conductivity of 1.0×10⁻¹² Siemens per meter at a temperature of 20.0° C. In one or more embodiments, a portion of each forceps arm 100 may be coated with a material having a thermal conductivity of less than 1.0 Watts per meter Kelvin at a temperature of 20.0° C., e.g., a portion of each forceps arm 100 may be coated with a material having a thermal conductivity of 0.25 Watts per meter Kelvin at a temperature of 20.0° C. Illustratively, a portion of each forceps arm 100 may be coated with a material having an electrical conductivity of less than 1.0×10⁻⁸ Siemens per meter and a thermal conductivity of less than 1.0 Watts per meter Kelvin at a temperature of 20.0° C., e.g., a portion of each forceps arm 100 may be coated with a material having an electrical conductivity of 1.0×10⁻¹² Siemens per meter and a thermal conductivity of 0.25 Watts per meter Kelvin at a temperature of 20.0° C. In one or more embodiments, a portion of each forceps arm 100 may be coated with a material wherein a coating thickness of the material is in a range of 0.005 to 0.008 inches, e.g., a portion of each forceps arm 100 may be coated with a material wherein a coating thickness of the material is 0.0065 inches. Illustratively, a portion of each forceps arm 100 may be coated with a material wherein a coating thickness of the material is less than 0.005 inches or greater than 0.008 inches. In one or more embodiments, a portion of each forceps arm 100 may be coated with a material having an electrical conductivity of less than 1.0×10⁻⁸ Siemens per meter and a thermal conductivity of less than 1.0 Watts per meter Kelvin at a temperature of 20.0° C. wherein a coating thickness of the material is in a range of 0.005 to 0.008 inches, e.g., a portion of each forceps arm 100 may be coated with a material having an electrical conductivity of 1.0×10⁻¹² Siemens per meter and a thermal conductivity of 0.25 Watts per meter Kelvin at a temperature of 20.0° C. wherein a coating thickness of the material is 0.0065 inches. Illustratively, a portion of each forceps arm 100 may be coated with a material having a material mass in a range of 0.0015 to 0.0025 pounds, e.g., a portion of each forceps arm 100 may be coated with a material having a material mass of 0.0021 pounds. In one or more embodiments, a portion of each forceps arm 100 may be coated with a material having a material mass less than 0.0015 pounds or greater than 0.0025 pounds.

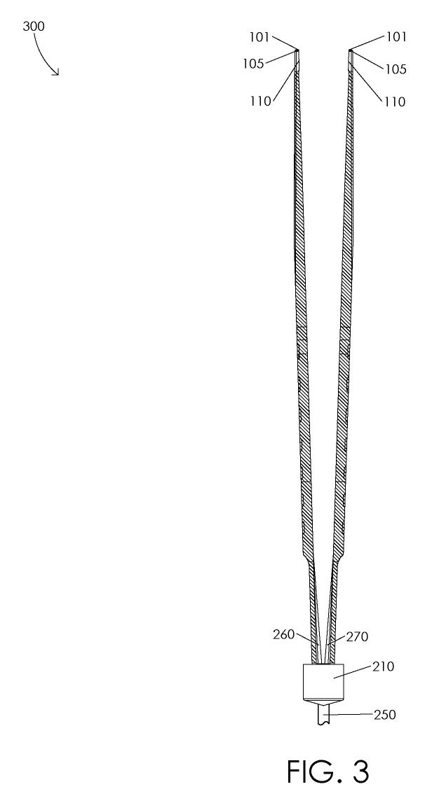

FIG. 3 is a schematic diagram illustrating an assembled temperature monitoring bipolar forceps 300. In one or more embodiments, an assembled temperature monitoring bipolar forceps 300 may comprise a first forceps arm 100 and a second forceps arm 100. Illustratively, forceps arms 100 may be fixed within forceps arm housings wherein forceps arm proximal ends 102 are fixed within input conductor isolation mechanism 210 and forceps arm distal ends 101 are separated by a maximum conductor tip 110 separation distance, e.g., forceps arm proximal ends 102 may be disposed in input conductor isolation mechanism distal end 211 wherein forceps arm distal ends 101 are separated by a maximum conductor tip 110 separation distance. In one or more embodiments, a surgeon may decrease a distance between first forceps arm distal end 101 and second forceps arm distal end 101, e.g., by applying a force to a lateral portion of forceps arms 100. Illustratively, a surgeon may decrease a distance between first forceps arm distal end 101 and second forceps arm distal end 101, e.g., until first forceps arm distal end 101 contacts second forceps arm distal end 101. In one or more embodiments, a contact between first forceps arm distal end 101 and second forceps arm distal end 101 may be configured to electrically connect conductor tips 110. Illustratively, an electrical connection of conductor tips 110 may be configured to close an electrical circuit. In one or more embodiments, a surgeon may increase a distance between first forceps arm distal end 101 and second forceps arm distal end 101, e.g., by reducing a force applied to a lateral portion of forceps arms 100. Illustratively, increasing a distance between first forceps arm distal end 101 and second forceps arm distal end 101 may be configured to separate conductor tips 110. In one or more embodiments, a separation of conductor tips 110 may be configured to open an electrical circuit.

Illustratively, a portion of housing sleeve interface 205 may be disposed in a portion of input conductor isolation mechanism 210, e.g., housing sleeve interface distal end 206 may be disposed in input conductor isolation mechanism proximal end 212. In one or more embodiments, a portion of housing sleeve interface 205 may be fixed in a portion of input conductor isolation mechanism 210, e.g., a portion of housing sleeve interface 205 may be fixed in a portion of input conductor isolation mechanism 210 by an adhesive, an epoxy, a friction fit, a snap-fit, a weld, a setscrew, etc. Illustratively, a portion of housing sleeve 250 may be disposed in a portion of housing sleeve interface 205, e.g., housing sleeve distal end 251 may be disposed in housing sleeve interface proximal end 207. In one or more embodiments, a portion of housing sleeve 250 may be fixed in a portion of housing sleeve interface 205, e.g., a portion of housing sleeve 250 may be fixed in a portion of housing sleeve interface 205 by an adhesive, an epoxy, a friction fit, a snap-fit, a weld, a setscrew, etc. Illustratively, a portion of machine connector 240 may be disposed in a portion of housing sleeve 250, e.g., machine connector proximal end 242 may be disposed in housing sleeve proximal end 252. In one or more embodiments, a portion of machine connector 240 may be fixed in a portion of housing sleeve 250, e.g., a portion of machine connector 240 may be fixed in a portion of housing sleeve 250 by an adhesive, an epoxy, a friction fit, a snap-fit, a weld, a setscrew, etc.

Illustratively, bipolar cord 230 may be disposed in housing sleeve 250, e.g., bipolar cord 230 may be disposed in housing sleeve 250 wherein a portion of bipolar cord 230 extends out from a portion of housing sleeve 250. In one or more embodiments, bipolar cord 230 may be disposed in housing sleeve 250 wherein first input conduct proximal end 231 extends from housing sleeve proximal end 252. Illustratively, bipolar cord 230 may be disposed in housing sleeve 250 wherein first input conductor distal end 232 extends from housing sleeve distal end 251. In one or more embodiments, bipolar cord 230 may be disposed in housing sleeve 250 wherein second input conduct proximal end 233 extends from housing sleeve proximal end 252. Illustratively, bipolar cord 230 may be disposed in housing sleeve 250 wherein second input conductor distal end 234 extends from housing sleeve distal end 251. In one or more embodiments, a portion of bipolar cord 230 may be disposed in a portion of machine connector 240, e.g., first input conductor proximal end 231 and second input conductor proximal end 233 may be disposed in a portion of machine connector proximal end 242. Illustratively, first input conductor proximal end 231 and second input conductor proximal end 233 may be fixed in machine connector 240, e.g., first input conductor proximal end 231 and second input conductor proximal end 233 may be fixed in machine connector 240 by an adhesive, an epoxy, a friction fit, a snap-fit, a weld, a setscrew, etc. In one or more embodiments, a portion of bipolar cord 230 may be electrically connected to machine connector 240, e.g., first input conductor proximal end 231 and second input conductor proximal end 233 may be electrically connected to machine connector.

Illustratively, a portion of bipolar cord 230 may be disposed in a portion of input conductor isolation mechanism 210, e.g., first input conductor distal end 232 and second input conductor distal end 234 may be disposed in input conductor isolation mechanism 210. In one or more embodiments, a portion of bipolar cord 230 may be fixed in a portion of input conductor isolation mechanism 210, e.g., a portion of bipolar cord 230 may be fixed in a portion of input conductor isolation mechanism 210 by an adhesive, an epoxy, a friction fit, a snap-fit, a weld, a setscrew, etc. Illustratively, a portion of bipolar cord 230 may be disposed in a portion of a first forceps arm 100 and a portion of bipolar cord 230 may be disposed in a portion of a second forceps arm 100, e.g., first input conductor distal end 232 may be disposed in a first input conductor housing 103 and second input conductor distal end 234 may be disposed in a second input conductor housing 103. In one or more embodiments, a portion of bipolar cord 230 may be electrically connected to a first forceps arm 100 and a portion of bipolar cord 230 may be electrically connected to a second forceps arm 100, e.g., first input conductor distal end 232 may be electrically connected to a first input conductor housing 103 and second input conductor distal end 234 may be electrically connected to a second input conductor housing 103.

Illustratively, first thermocouple wire 260 may be disposed in housing sleeve 250 wherein a portion of first thermocouple wire 260 extends from a portion of housing sleeve 250. In one or more embodiments, first thermocouple wire 260 may be disposed in housing sleeve 250 wherein first thermocouple wire distal end 261 extends out from housing sleeve distal end 251. Illustratively, first thermocouple wire 260 may be disposed in housing sleeve 250 wherein first thermocouple wire proximal end 262 extends out from housing sleeve proximal end 252. In one or more embodiments, a portion of first thermocouple wire 260 may be disposed in a portion of machine connector 240, e.g., first thermocouple wire proximal end 262 may be disposed in machine connector proximal end 242. Illustratively, a portion of first thermocouple wire 260 may be electrically connected to machine connector 240, e.g., first thermocouple wire proximal end 262 may be electrically connected to machine connector 240. In one or more embodiments, a portion of first thermocouple wire 260 may be disposed in a portion of a first forceps arm 100, e.g., a portion of first thermocouple wire 260 may be disposed in a first proximal channel 160. Illustratively, a portion of first thermocouple wire 260 may be fixed in a first proximal channel 160, e.g., a portion of first thermocouple wire 260 may be fixed in a first proximal channel 160 by an adhesive, an epoxy, a friction fit, a snap-fit, a weld, a setscrew, etc. In one or more embodiments, a portion of first thermocouple wire 260 may be disposed in a first distal channel 180. Illustratively, a portion of first thermocouple wire 260 may be fixed in a first distal channel 180, e.g., a portion of first thermocouple wire 260 may be fixed in a first distal channel 180 by an adhesive, an epoxy, a friction fit, a snap-fit, a weld, a setscrew, etc. In one or more embodiments, a portion of first thermocouple wire 260 may be disposed in a portion of a first forceps arm 100 wherein first thermocouple 265 is disposed in a first thermocouple housing 105. Illustratively, first thermocouple 265 may be fixed in a first thermocouple housing 105, e.g., first thermocouple 265 may be fixed in a first thermocouple housing 105 by an adhesive, an epoxy, a friction fit, a snap-fit, a weld, a setscrew, etc.

Illustratively, second thermocouple wire 270 may be disposed in housing sleeve 250 wherein a portion of second thermocouple wire 270 extends from a portion of housing sleeve 250. In one or more embodiments, second thermocouple wire 270 may be disposed in housing sleeve 250 wherein second thermocouple wire distal end 271 extends out from housing sleeve distal end 251. Illustratively, second thermocouple wire 270 may be disposed in housing sleeve 250 wherein second thermocouple wire proximal end 272 extends out from housing sleeve proximal end 252. In one or more embodiments, a portion of second thermocouple wire 270 may be disposed in a portion of machine connector 240, e.g., second thermocouple wire proximal end 272 may be disposed in machine connector proximal end 242. Illustratively, a portion of second thermocouple wire 270 may be electrically connected to machine connector 240, e.g., second thermocouple wire proximal end 272 may be electrically connected to machine connector 240. In one or more embodiments, a portion of second thermocouple wire 270 may be disposed in a portion of a second forceps arm 100, e.g., a portion of second thermocouple wire 270 may be disposed in a second proximal channel 160. Illustratively, a portion of second thermocouple wire 270 may be fixed in a second proximal channel 160, e.g., a portion of second thermocouple wire 270 may be fixed in a second proximal channel 160 by an adhesive, an epoxy, a friction fit, a snap-fit, a weld, a setscrew, etc. In one or more embodiments, a portion of second thermocouple wire 270 may be disposed in a second distal channel 180. Illustratively, a portion of second thermocouple wire 270 may be fixed in a second distal channel 180, e.g., a portion of second thermocouple wire 270 may be fixed in a second distal channel 180 by an adhesive, an epoxy, a friction fit, a snap-fit, a weld, a setscrew, etc. In one or more embodiments, a portion of second thermocouple wire 270 may be disposed in a portion of a second t forceps arm 100 wherein second thermocouple 275 is disposed in a second thermocouple housing 105. Illustratively, second thermocouple 275 may be fixed in a second thermocouple housing 105, e.g., second thermocouple 275 may be fixed in a second thermocouple housing 105 by an adhesive, an epoxy, a friction fit, a snap-fit, a weld, a setscrew, etc.

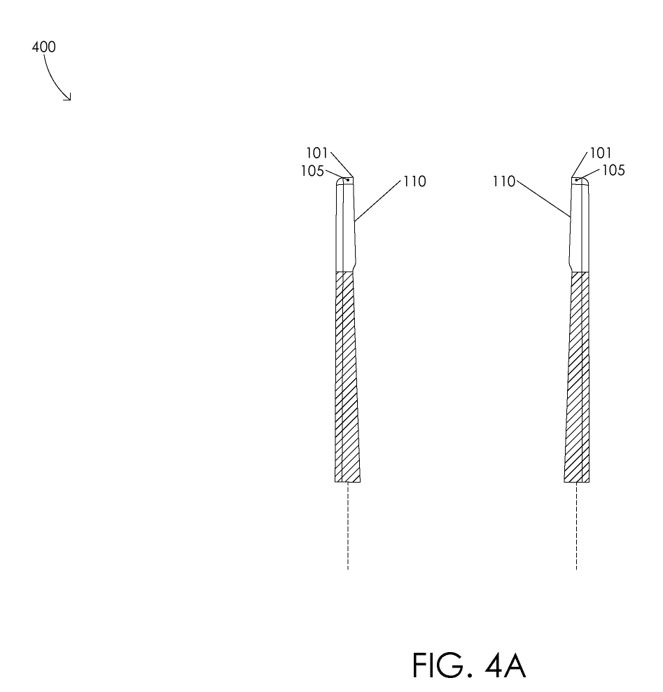

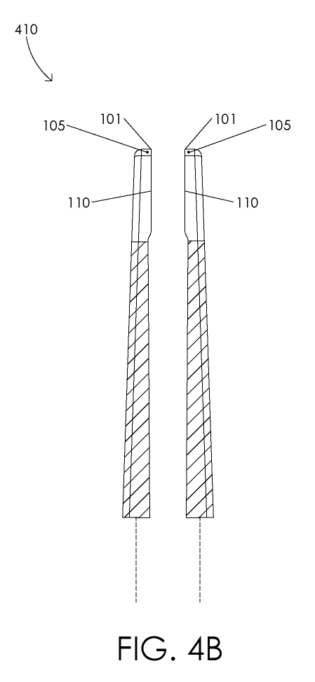

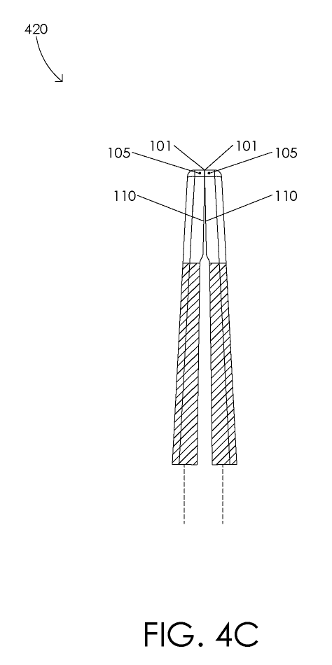

FIGS. 4A, 4B, and 4C are schematic diagrams illustrating a gradual closing of a temperature monitoring bipolar forceps. FIG. 4A illustrates conductor tips in an open orientation 400. Illustratively, conductor tips 110 may comprise conductor tips in an open orientation 400, e.g., when forceps arm distal ends 101 are separated by a maximum conductor tip 110 separation distance. In one or more embodiments, forceps arm distal ends 101 may be separated by a distance in a range of 0.5 to 0.7 inches when conductor tips 110 comprise conductor tips in an open orientation 400, e.g., forceps arm distal ends 101 may be separated by a distance of 0.625 inches when conductor tips 110 comprise conductor tips in an open orientation 400. Illustratively, forceps arm distal ends 101 may be separated by a distance less than 0.5 inches or greater than 0.7 inches when conductor tips 110 comprise conductor tips in an open orientation 400. In one or more embodiments, conductor tips 110 may comprise conductor tips in an open orientation 400, e.g., when no force is applied to a lateral portion of forceps arms 100.

FIG. 4B illustrates conductor tips in a partially closed orientation 410. Illustratively, an application of a force to a lateral portion of forceps arms 100 may be configured to gradually close conductor tips 110 from conductor tips in an open orientation 400 to conductor tips in a partially closed orientation 410. In one or more embodiments, an application of a force to a lateral portion of forceps arms 100 may be configured to decrease a distance between first forceps arm distal end 101 and second forceps arm distal end 101. Illustratively, an application of a force having a magnitude in a range of 0.05 to 0.3 pounds to a lateral portion of forceps arms 100 may be configured to decrease a distance between first forceps arm distal end 101 and second forceps arm distal end 101, e.g., an application of a force having a magnitude of 0.2 pounds to a lateral portion of forceps arms 100 may be configured to decrease a distance between first forceps arm distal end 101 and second forceps arm distal end 101. In one or more embodiments, an application of a force having a magnitude less than 0.05 pounds or greater than 0.3 pounds to a lateral portion of forceps arms 100 may be configured to decrease a distance between first forceps arm distal end 101 and second forceps arm distal end 101. Illustratively, a decrease of a distance between first forceps arm distal end 101 and second forceps arm distal end 101 may be configured to decrease a distance between conductor tips 110. In one or more embodiments, an application of a force having a magnitude in a range of 0.05 to 0.3 pounds to a lateral portion of forceps arms 100 may be configured to gradually close conductor tips 110 from conductor tips in an open orientation 400 to conductor tips in a partially closed orientation 410. Illustratively, an application of a force having a magnitude less than 0.05 pounds or greater than 0.3 pounds to a lateral portion of forceps arms 100 may be configured to gradually close conductor tips 110 from conductor tips in an open orientation 400 to conductor tips in a partially closed orientation 410. In one or more embodiments, an amount of force applied to a lateral portion of forceps arms 100 configured to close conductor tips 110 to conductor tips in a partially closed orientation 410 and a total mass of a bipolar forceps with active cooling may have a force applied to total mass ratio in a range of 1.25 to 8.75, e.g., an amount of force applied to a lateral portion of forceps arms 100 configured to close conductor tips 110 to conductor tips in a partially closed orientation 410 and a total mass of a bipolar forceps with active cooling may have a force applied to total mass ratio of 5.25. Illustratively, an amount of force applied to a lateral portion of forceps arms 100 configured to close conductor tips 110 to conductor tips in a partially closed orientation 410 and a total mass of a bipolar forceps with active cooling may have a force applied to total mass ratio less than 1.25 or greater than 8.75.

In one or more embodiments, a surgeon may dispose a tissue between a first forceps arm conductor tip 110 and a second forceps arm conductor tip 110, e.g., a surgeon may dispose a tumor tissue between a first forceps arm conductor tip 110 and a second forceps arm conductor tip 110. Illustratively, disposing a tissue between a first forceps arm conductor tip 110 and a second forceps arm conductor tip 110 may be configured to electrically connect the first forceps arm conductor tip 110 and the second forceps arm conductor tip 110, e.g., the tissue may electrically connect the first forceps arm conductor tip 110 and the second forceps arm conductor tip 110. In one or more embodiments, electrically connecting a first forceps arm conductor tip 110 and a second forceps arm conductor tip 110 may be configured to supply an electrical current to a tissue. Illustratively, supplying an electrical current to a tissue may be configured to coagulate the tissue, cauterize the tissue, ablate the tissue, etc. In one or more embodiments, electrically connecting a first forceps arm conductor tip 110 and a second forceps arm conductor tip 110 may be configured to seal a vessel, induce hemostasis, etc.

Illustratively, coagulating a tissue, cauterizing a tissue, ablating a tissue, sealing a vessel, or inducing hemostasis may be configured to increase a temperature of a first conductor tip 110. In one or more embodiments, first thermocouple 265 may be configured to measure a temperature of a first conductor tip 110, e.g., first thermocouple 265 may be configured to continuously measure a temperature of a first conductor tip 110. Illustratively, a system control 800 of an electrosurgical generator may be configured to visually display a temperature measured at a first conductor tip 110 to a user. In one or more embodiments, a system control 800 of an electrosurgical generator may be configured to audibly communicate a temperature measured at a first conductor tip 110 to a user. Illustratively, a system control 800 of an electrosurgical generator may be configured to communicate to a user that a second temperature measured by first thermocouple 265 is greater than a first temperature measured by first thermocouple 265, e.g., a system control 800 of an electrosurgical generator may be configured to communicate to a user that a temperature of a first conductor tip 110 is increasing. In one or more embodiments, a system control 800 of an electrosurgical generator may be configured to communicate to a user that a second temperature measured by first thermocouple 265 is less than a first temperature measured by first thermocouple 265, e.g., a system control 800 of an electrosurgical generator may be configured to communicate to a user that a temperature of a first conductor tip 110 is decreasing. Illustratively, a system control 800 of an electrosurgical generator may be configured to communicate to a user that a temperature measured at a first conductor tip 110 is greater than 70.0 degrees Celsius. In one or more embodiments, a system control 800 of an electrosurgical generator may be configured to prompt a user to input a maximum temperature of a first conductor tip 110. Illustratively, the system control 800 of the electrosurgical generator may be configured to communicate to the user that a temperature measured by first thermocouple 265 is greater than or equal to the maximum temperature of first conductor tip 110. In one or more embodiments, the system control 800 of the electrosurgical generator may be configured to reduce an amount of power supplied to first conductor tip 110 in response to a measurement of a temperature by first thermocouple 265 that is greater than or equal to the maximum temperature of first conductor tip 110.

Illustratively, coagulating a tissue, cauterizing a tissue, ablating a tissue, sealing a vessel, or inducing hemostasis may be configured to increase a temperature of a second conductor tip 110. In one or more embodiments, second thermocouple 275 may be configured to measure a temperature of a second conductor tip 110, e.g., second thermocouple 275 may be configured to continuously measure a temperature of a second conductor tip 110. Illustratively, a system control 800 of an electrosurgical generator may be configured to visually display a temperature measured at a second conductor tip 110 to a user. In one or more embodiments, a system control 800 of an electrosurgical generator may be configured to audibly communicate a temperature measured at a second conductor tip 110 to a user. Illustratively, a system control 800 of an electrosurgical generator may be configured to communicate to a user that a second temperature measured by second thermocouple 275 is greater than a first temperature measured by second thermocouple 275, e.g., a system control 800 of an electrosurgical generator may be configured to communicate to a user that a temperature of a second conductor tip 110 is increasing. In one or more embodiments, a system control 800 of an electrosurgical generator may be configured to communicate to a user that a second temperature measured by second thermocouple 275 is less than a first temperature measured by second thermocouple 275, e.g., a system control 800 of an electrosurgical generator may be configured to communicate to a user that a temperature of a second conductor tip 110 is decreasing. Illustratively, a system control 800 of an electrosurgical generator may be configured to communicate to a user that a temperature measured at a second conductor tip 110 is greater than 70.0 degrees Celsius. In one or more embodiments, a system control 800 of an electrosurgical generator may be configured to prompt a user to input a maximum temperature of a second conductor tip 110. Illustratively, the system control 800 of the electrosurgical generator may be configured to communicate to the user that a temperature measured by second thermocouple 275 is greater than or equal to the maximum temperature of second conductor tip 110. In one or more embodiments, the system control 800 of the electrosurgical generator may be configured to reduce an amount of power supplied to second conductor tip 110 in response to a measurement of a temperature by second thermocouple 275 that is greater than or equal to the maximum temperature of second conductor tip 110.

Illustratively, first thermocouple 265 may be configured to measure a temperature of a first conductor tip 110 and second thermocouple 275 may be configured to measure a temperature of a second conductor tip 110. In one or more embodiments, a system control 800 of an electrosurgical generator may be configured to receive a first signal corresponding to a temperature measured by first thermocouple 265 and a second signal corresponding to a temperature measured by second thermocouple 275. Illustratively, the system control 800 of the electrosurgical generator may be configured to compare a temperature measured by first thermocouple 265 and a temperature measured by second thermocouple 275. In one or more embodiments, if the system control 800 of the electrosurgical generator determines that a temperature measured by first thermocouple 265 is greater than a temperature measured by second thermocouple 275, then the system control 800 of the electrosurgical generator may be configured to visually display a temperature measured at a first conductor tip 110 to a user. Illustratively, if the system control 800 of the electrosurgical generator determines that a temperature measured by first thermocouple 265 is greater than a temperature measured by second thermocouple 275, then the system control 800 of the electrosurgical generator may be configured to audibly communicate a temperature measured at a first conductor tip 110 to a user. In one or more embodiments, if the system control 800 of the electrosurgical generator determines that a current temperature measured by first thermocouple 265 is greater than a current temperature measured by second thermocouple 275 and if the system control 800 of the electrosurgical generator determines that the temperature measured by first thermocouple 265 is greater than a previous temperature measured by first thermocouple 265 and greater than a previous temperature measured by second thermocouple 275, then the system control 800 of the electrosurgical generator may be configured to communicate to a user that a temperature at a surgical site is increasing. Illustratively, if the system control 800 of the electrosurgical generator determines that a current temperature measured by first thermocouple 265 is greater than a current temperature measured by second thermocouple 275 and if the system control 800 of the electrosurgical generator determines that the temperature measured by first thermocouple 265 is less than a previous temperature measured by first thermocouple 265 and less than a previous temperature measured by second thermocouple 275, then the system control 800 of the electrosurgical generator may be configured to communicate to a user that a temperature at a surgical site is decreasing. In one or more embodiments, if the system control 800 of the electrosurgical generator determines that a temperature measured by first thermocouple 265 is greater than a temperature measured by second thermocouple 275 and if the system control 800 of the electrosurgical generator determines that the temperature measured by first thermocouple 265 is greater than 70.0 degrees Celsius, then the system control 800 of the electrosurgical generator may be configured to communicate to a user that a temperature at a surgical site is greater than 70.0 degrees Celsius. Illustratively, the system control 800 of the electrosurgical generator may be configured to prompt a user to input a maximum temperature at a surgical site. In one or more embodiments, if the system control 800 of the electrosurgical generator determines that a temperature measured by first thermocouple 265 is greater than a temperature measured by second thermocouple 275 and if the system control 800 of the electrosurgical generator determines that the temperature measured by first thermocouple 265 is greater than or equal to the maximum temperature at a surgical site, then the system control 800 of the electrosurgical generator may be configured to communicate to a user that a temperature at a surgical site is greater than or equal to the maximum temperature at a surgical site.

In one or more embodiments, if the system control 800 of the electrosurgical generator determines that a temperature measured by second thermocouple 275 is greater than a temperature measured by first thermocouple 265, then the system control 800 of the electrosurgical generator may be configured to visually display a temperature measured at a second conductor tip 110 to a user. Illustratively, if the system control 800 of the electrosurgical generator determines that a temperature measured by second thermocouple 275 is greater than a temperature measured by first thermocouple 265, then the system control 800 of the electrosurgical generator may be configured to audibly communicate a temperature measured at a second conductor tip 110 to a user. In one or more embodiments, if the system control 800 of the electrosurgical generator determines that a current temperature measured by second thermocouple 275 is greater than a current temperature measured by first thermocouple 265 and if the system control 800 of the electrosurgical generator determines that the temperature measured by second thermocouple 275 is greater than a previous temperature measured by second thermocouple 275 and greater than a previous temperature measured by first thermocouple 265, then the system control 800 of the electrosurgical generator may be configured to communicate to a user that a temperature at a surgical site is increasing. Illustratively, if the system control 800 of the electrosurgical generator determines that a current temperature measured by second thermocouple 275 is greater than a current temperature measured by first thermocouple 265 and if the system control 800 of the electrosurgical generator determines that the temperature measured by second thermocouple 275 is less than a previous temperature measured by second thermocouple 275 and less than a previous temperature measured by first thermocouple 265, then the system control 800 of the electrosurgical generator may be configured to communicate to a user that a temperature at a surgical site is decreasing. In one or more embodiments, if the system control 800 of the electrosurgical generator determines that a temperature measured by second thermocouple 275 is greater than a temperature measured by first thermocouple 265 and if the system control 800 of the electrosurgical generator determines that the temperature measured by second thermocouple 275 is greater than 70.0 degrees Celsius, then the system control 800 of the electrosurgical generator may be configured to communicate to a user that a temperature at a surgical site is greater than 70.0 degrees Celsius. Illustratively, the system control 800 of the electrosurgical generator may be configured to prompt a user to input a maximum temperature at a surgical site. In one or more embodiments, if the system control 800 of the electrosurgical generator determines that a temperature measured by second thermocouple 275 is greater than a temperature measured by first thermocouple 265 and if the system control 800 of the electrosurgical generator determines that the temperature measured by second thermocouple 275 is greater than or equal to the maximum temperature at a surgical site, then the system control 800 of the electrosurgical generator may be configured to communicate to a user that a temperature at a surgical site is greater than or equal to the maximum temperature at a surgical site.

FIG. 4C illustrates conductor tips in a fully closed orientation 420. Illustratively, an application of a force to a lateral portion of forceps arms 100 may be configured to gradually close conductor tips 110 from conductor tips in a partially closed orientation 410 to conductor tips in a fully closed orientation 420. In one or more embodiments, first forceps arm conductor tip 110 and second forceps arm conductor tip 110 may have a contact area in a range of 0.01 to 0.015 square inches when conductor tips 110 comprise conductor tips in a fully closed orientation 420, e.g., first forceps arm conductor tip 110 and second forceps arm conductor tip 110 may have a contact area of 0.0125 square inches when conductor tips 110 comprise conductor tips in a fully closed orientation 420. Illustratively, first forceps arm conductor tip 110 and second forceps arm conductor tip 110 may have a contact area less than 0.01 square inches or greater than 0.015 square inches when conductor tips 110 comprise conductor tips in a fully closed orientation 420. Illustratively, an application of a force having a magnitude in a range of 1.5 to 3.3 pounds to a lateral portion of forceps arms 100 may be configured to gradually close conductor tips 110 from conductor tips in a partially closed orientation 410 to conductor tips in a fully closed orientation 420, e.g., an application of a force having a magnitude of 2.5 pounds to a lateral portion of forceps arms may be configured to gradually close conductor tips 110 from conductor tips in a partially closed orientation 410 to conductor tips in a fully closed orientation 420. In one or more embodiments, an application of a force having a magnitude less than 1.5 pounds or greater than 3.3 pounds to a lateral portion of forceps arms 100 may be configured to gradually close conductor tips 110 from conductor tips in a partially closed orientation 410 to conductor tips in a fully closed orientation 420.

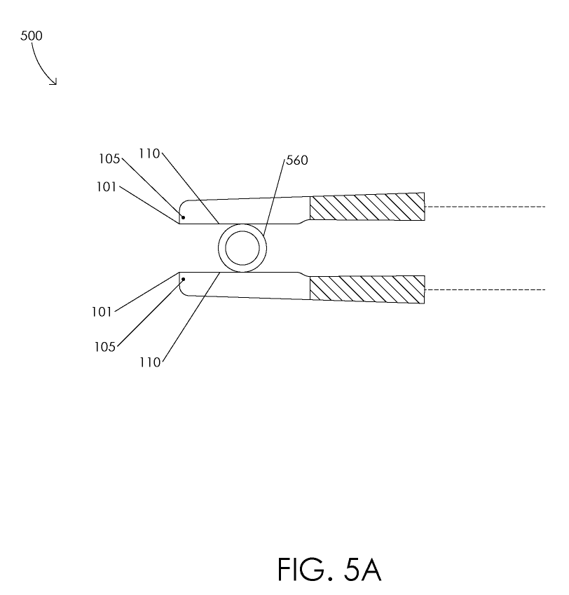

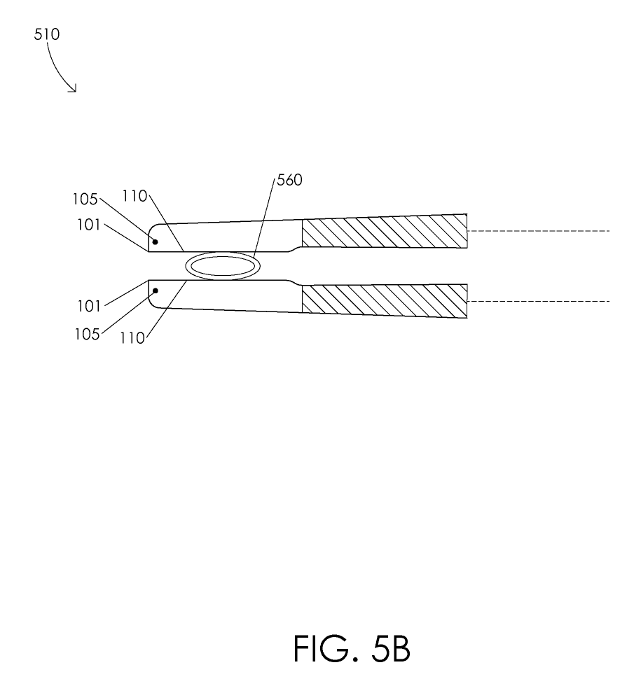

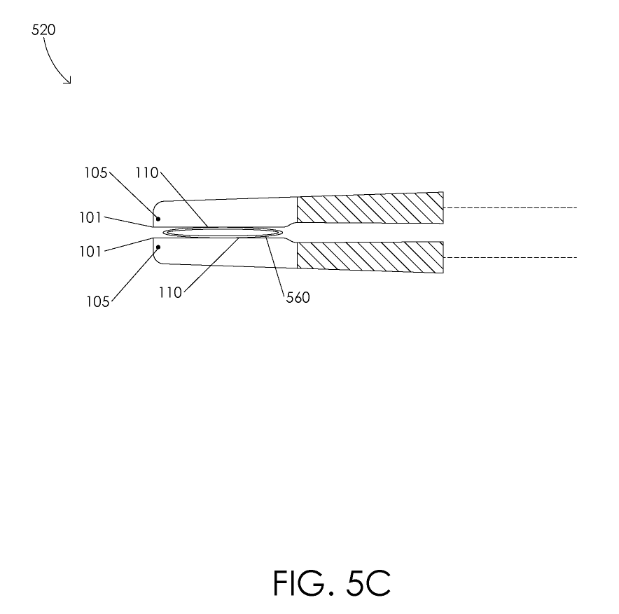

FIGS. 5A, 5B, and 5C are schematic diagrams illustrating a uniform compression of a vessel 560. In one or more embodiments, vessel 560 may comprise a blood vessel of an arteriovenous malformation. FIG. 5A illustrates an uncompressed vessel 500. Illustratively, vessel 560 may comprise an uncompressed vessel 500, e.g., when vessel 560 has a natural geometry. In one or more embodiments, vessel 560 may comprise an uncompressed vessel, e.g., when conductor tips 110 comprise conductor tips in a partially closed orientation 410. Illustratively, a surgeon may dispose vessel 560 between a first conductor tip 110 and a second conductor tip 110, e.g., when conductor tips 110 comprise conductor tips in an open orientation 400. In one or more embodiments, an application of a force to a lateral portion of forceps arms 100 may be configured to gradually close conductor tips 110 from conductor tips in an open orientation 400 to conductor tips in a partially closed orientation 410. Illustratively, vessel 560 may electrically connect a first conductor tip 110 and a second conductor tip 110, e.g., when vessel 560 comprises an uncompressed vessel 500. In one or more embodiments, a surgeon may identify an orientation of conductor tips 110 wherein conductor tips 110 initially contact vessel 560. Illustratively, a geometry of forceps arms 100 may be configured to allow a surgeon to visually identify an orientation of conductor tips 110 wherein conductor tips 110 initially contact vessel 560. In one or more embodiments, a mass of forceps arms 100 may be configured to allow a surgeon to tactilely identify an orientation of conductor tips 110 wherein conductor tips 110 initially contact vessel 560. Illustratively, a geometry of forceps arms 100 and a mass of forceps arms 100 may be configured to allow a surgeon to both visually and tactilely identify an orientation of conductor tips 110 wherein conductor tips 110 initially contact vessel 560.

FIG. 5B illustrates a partially compressed vessel 510. Illustratively, an application of a force to a lateral portion of forceps arms 100 may be configured to uniformly compress vessel 560 from an uncompressed vessel 500 to a partially compressed vessel 510. In one or more embodiments, an application of a force to a lateral portion of forceps arms 100 may be configured to uniformly increase a contact area between vessel 560 and forceps arm conductor tips 110. Illustratively, vessel 560 may electrically connect first forceps arm conductor tip 110 and second forceps arm conductor tip 110, e.g., when vessel 560 comprises a partially compressed vessel 510. In one or more embodiments, an application of a force to a lateral portion of forceps arms 100 may be configured to compress vessel 560 wherein vessel 560 maintains a symmetrical geometry with respect to a medial axis of vessel 560. Illustratively, vessel 560 may have a symmetrical geometry with respect to a medial axis of vessel 560 when vessel 560 comprises a partially compressed vessel 510. In one or more embodiments, conductor tips 110 may be configured to compress vessel 560 wherein no portion of vessel 560 is compressed substantially more than another portion of vessel 560, e.g., conductor tips 110 may be configured to evenly compress vessel 560 without pinching a first portion of vessel 560 or bulging a second portion of vessel 560. Illustratively, vessel 560 may be evenly compressed when vessel 560 comprises a partially compressed vessel 510.

FIG. 5C illustrates a fully compressed vessel 520. Illustratively, an application of a force to a lateral portion of forceps arms 100 may be configured to uniformly compress vessel 560 from a partially compressed vessel 510 to a fully compressed vessel 520. In one or more embodiments, an application of a force to a lateral portion of forceps arms 100 may be configured to uniformly increase a contact area between vessel 560 and forceps arm conductor tips 110. Illustratively, vessel 560 may electrically connect first forceps arm conductor tip 110 and second forceps arm conductor tip 110, e.g., when vessel 560 comprises a fully compressed vessel 520. In one or more embodiments, a surgeon may uniformly cauterize vessel 560, e.g., when vessel 560 comprises a fully compressed vessel 520. Illustratively, a surgeon may uniformly achieve hemostasis of vessel 560, e.g., when vessel 560 comprises a fully compressed vessel 520. In one or more embodiments, an application of a force to a lateral portion of forceps arms 100 may be configured to compress vessel 560 wherein vessel 560 maintains a symmetrical geometry with respect to a medial axis of vessel 560. Illustratively, vessel 560 may have a symmetrical geometry with respect to a medial axis of vessel 560 when vessel 560 comprises a fully compressed vessel 520. In one or more embodiments, conductor tips 110 may be configured to compress vessel 560 wherein no portion of vessel 560 is compressed substantially more than another portion of vessel 560, e.g., conductor tips 110 may be configured to evenly compress vessel 560 without pinching a first portion of vessel 560 or bulging a second portion of vessel 560. Illustratively, vessel 560 may be evenly compressed when vessel 560 comprises a fully compressed vessel 520.

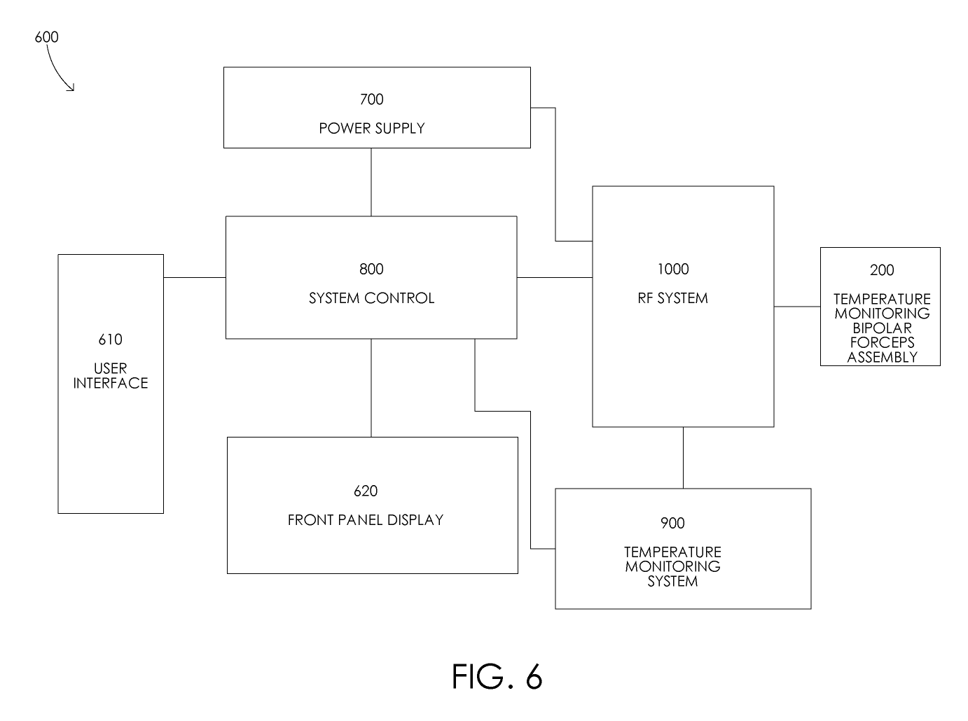

FIG. 6 is a schematic block diagram illustrating a temperature monitoring electrosurgical system 600. Illustratively, a temperature monitoring electrosurgical system 600 may comprise a user interface 610, a front panel display 620, a power supply 700, a system control 800, a temperature monitoring system, 900, an RF system 1000, and a temperature monitoring bipolar forceps assembly 200. Temperature monitoring electrosurgical system 600 comprises an electrosurgical generator. The electrosurgical generator comprises power supply 700, system control 800, and RF system 1000. In one or more embodiments, user interface 610 may be configured to accept user inputs. Illustratively, user interface 610 may be configured to communicate information to system control 800. In one or more embodiments, user interface 610 may be configured to receive a cauterization power setting (P_{SET}) and a cauterization temperature setting (T_{SET}) from a user. Illustratively, user interface 610 may be configured to communicate a cauterization power setting (P_{SET}) and a cauterization temperature setting (T_{SET}) to system control 800. In one or more embodiments, user interface 610 may comprise a footswitch configured to adjust one or more properties of temperature monitoring electrosurgical system 600. Illustratively, front panel display 620 may be configured to display information. In one or more embodiments, front panel display 620 may be configured to communicate information to system control 800. Illustratively, front panel display 620 may comprise a touchscreen configured to control one or more properties of temperature monitoring electrosurgical system 600.

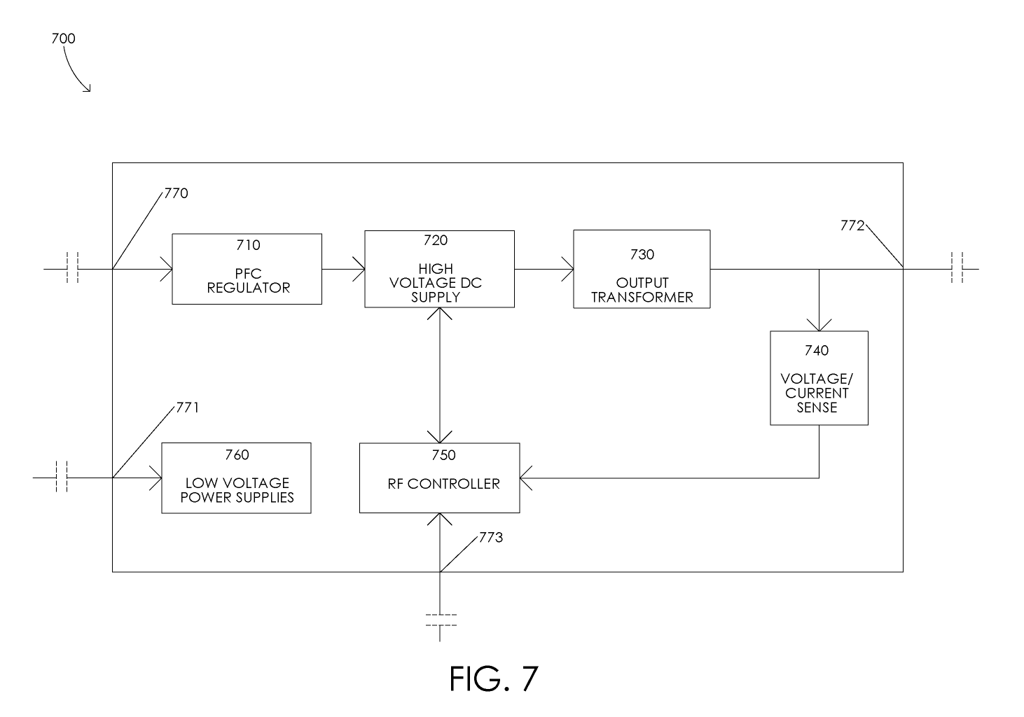

FIG. 7 is a schematic block diagram illustrating a power supply 700. Illustratively, power supply 700 may comprise a PFC regulator 710, a high voltage DC supply 720, an output transformer 730, a voltage/current sense 740, an RF controller 750, and low voltage power supplies 760. In one or more embodiments, PFC regulator 710 may be configured to correct a power factor of an AC mains input, e.g., PFC regulator 710 may be configured to correct a power factor of an AC mains input to electrical connection 770. Illustratively, low voltage power supplies 760 may be configured to supply power to temperature monitoring electrosurgical system 600 components, e.g., low voltage power supplies 760 may be configured to power a touchscreen. For example, low voltage power supplies 760 may be configured to receive a power input from an AC/DC converter from an AC mains input to electrical connection 771. In one or more embodiments, high voltage DC supply 720 and output transformer 730 may be configured to supply power to RF system 1000, e.g., high voltage DC supply 720 and output transformer 730 may be configured to supply power to RF system 1000 via electrical connection 772. Illustratively, voltage/current sense 740 may be configured to provide information to RF controller 750.

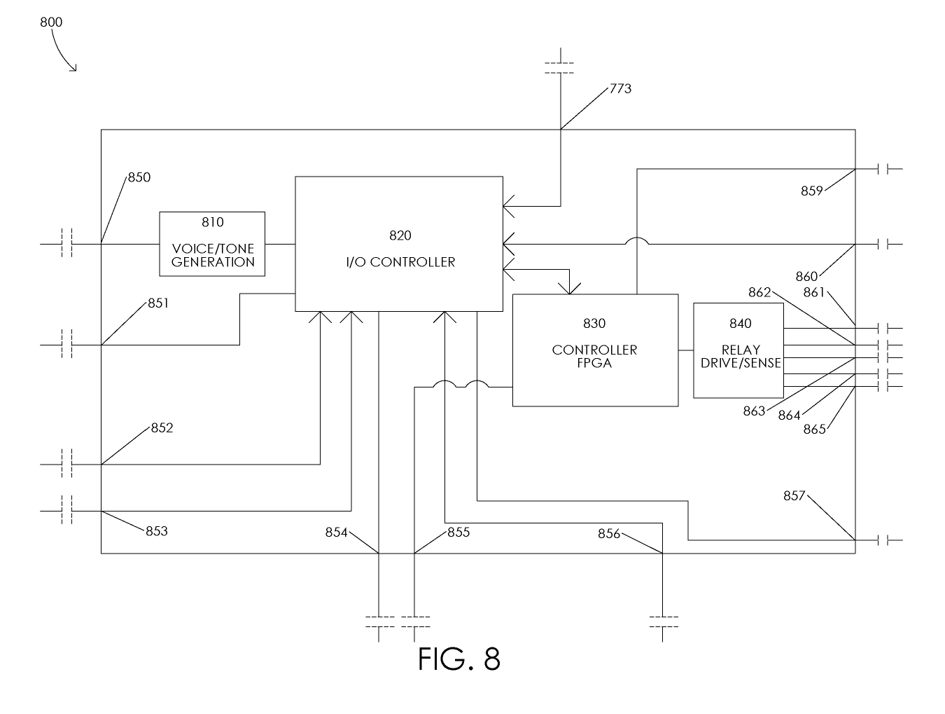

FIG. 8 is a schematic block diagram illustrating a system control 800. Illustratively, system control 800 may comprise a voice/tone generation 810, an I/O controller 820, a controller FPGA 830, and a relay drive/sense 840. In one or more embodiments, voice/tone generation 810 may be configured to convert audio user inputs into electrical signals. For example, a user may initiate a voice command to temperature monitoring electrosurgical system 600. Illustratively, I/O controller 820 may be configured to receive user inputs and control temperature monitoring electrosurgical system 600 outputs.

In one or more embodiments, I/O controller 820 may receive user inputs via electrical connections 850, 851, 852, or 853. Illustratively, I/O controller 820 may be configured to receive information related to one or more properties of temperature monitoring electrosurgical system 600, e.g., I/O controller 820 may be configured to receive information from RF system 1000. In one or more embodiments, I/O controller 820 may be configured to communicate with controller FPGA 830 to adjust one or more properties of temperature monitoring electrosurgical system 600.

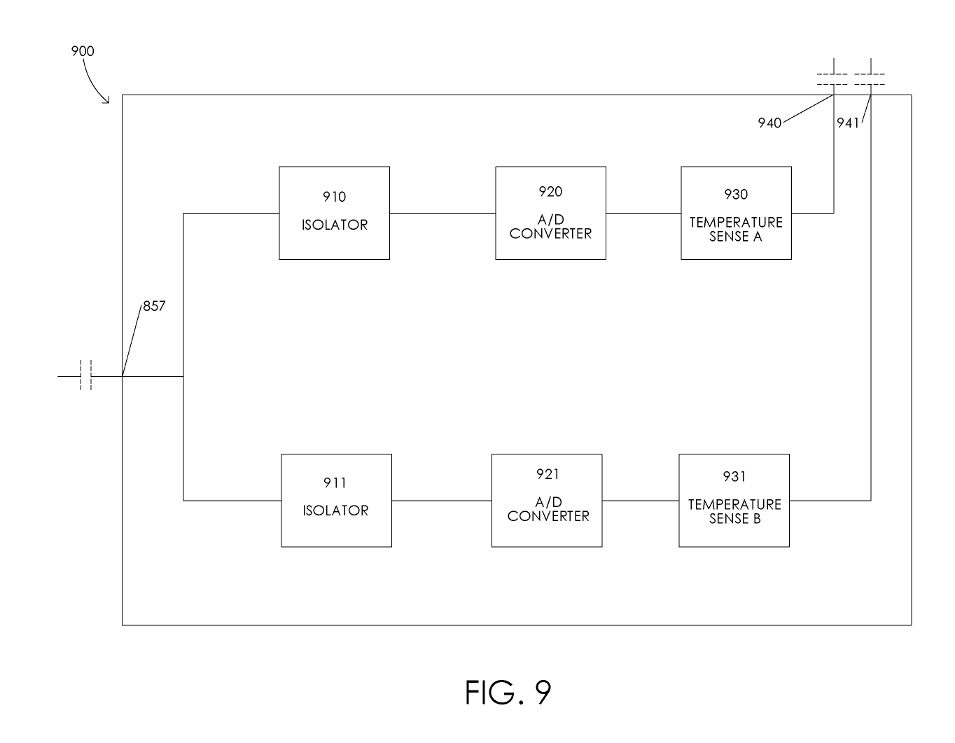

FIG. 9 is a schematic block diagram illustrating a temperature monitoring system 900. Illustratively, a temperature monitoring system 900 may comprise a first isolator 910, a second isolator 911, a first A/D converter 920, a second A/D converter, a first temperature sense 930, and a second temperature sense 931. In one or more embodiments, temperature monitoring system 900 may be configured to communicate data to system control 800 via electrical connection 857. Illustratively, temperature monitoring system 900 may be configured to receive data from system control 800 via electrical connection 857. In one or more embodiments, first thermocouple 265 may be electrically connected to first temperature sense 930 by electrical connection 940. Illustratively, first temperature sense 930 may be configured to receive a first signal corresponding to a voltage between first wire conductor 266 and second wire conductor 268. In one or more embodiments, first temperature sense 930 and first A/D converter 920 may be configured to convert the first signal into a first measured temperature. Illustratively, first isolator 910 may be configured to electrically isolate electrical connection 940 from electrical connection 857, e.g., first isolator 910 may be configured to prevent a ground loop from forming through a patient. In one or more embodiments, first isolator 910 may be configured to electrically isolate conductor tip 110 from system control 800. Illustratively, first isolator 910 may comprise an opto-isolator, e.g., first isolator 910 may comprise an LED and a sensor separated by a dielectric barrier. In one or more embodiments, second thermocouple 275 may be electrically connected to second temperature sense 931 by electrical connection 941. Illustratively, second temperature sense 931 may be configured to receive a second signal corresponding to a voltage between third wire conductor 276 and fourth wire conductor 278. In one or more embodiments, second temperature sense 931 and second A/D converter 921 may be configured to convert the second signal into a second measured temperature. Illustratively, second isolator 911 may be configured to electrically isolate electrical connection 941 from electrical connection 857, e.g., second isolator 911 may be configured to prevent a ground loop from forming through a patient. In one or more embodiments, second isolator 911 may be configured to electrically isolate conductor tip 110 from system control 800. Illustratively, second isolator 911 may comprise an opto-isolator, e.g., second isolator 911 may comprise an LED and a sensor separated by a dielectric barrier.

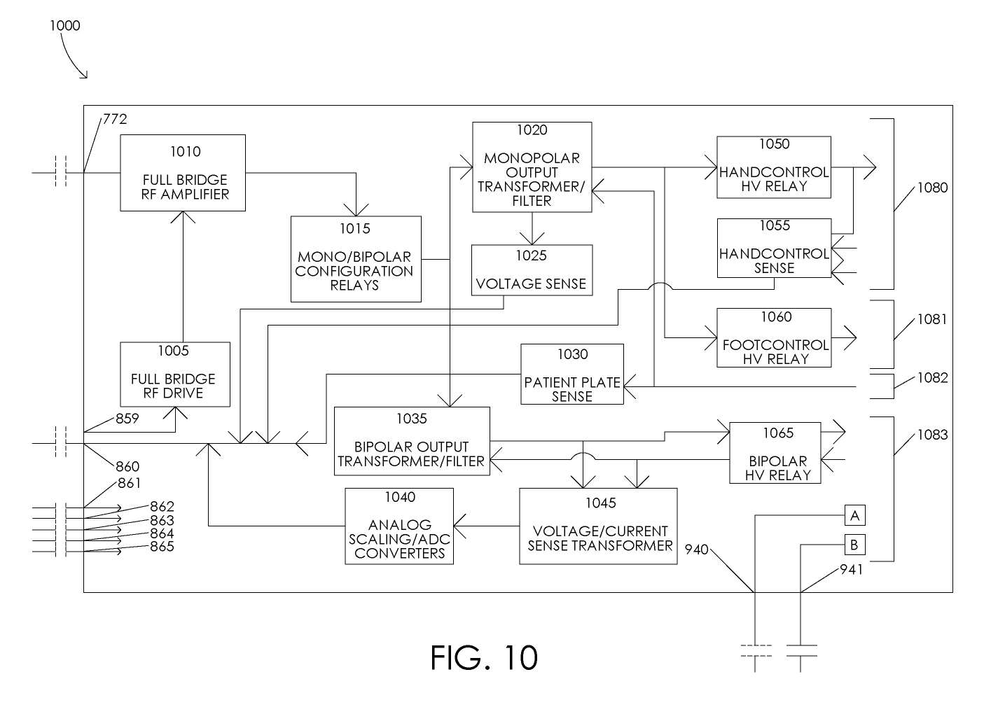

FIG. 10 is a schematic block diagram illustrating an RF system 1000. Illustratively, RF system 1000 may comprise a full bridge RF drive 1005, a full bridge RF amplifier 1010, mono/bipolar configuration relays 1015, a monopolar output transformer/filter 1020, a voltage sense 1025, a patient plate sense 1030, a bipolar output transformer/filter 1035, analog scaling ADC converters 1040, a voltage/current sense transformer 1045, a hand control HV relay 1050, a hand control sense 1055, a foot control HV relay 1060, and a bipolar HV relay 1065. In one or more embodiments, full bridge RF drive 1005 and full bridge RF amplifier 1010 may be configured to control a frequency and amplitude of a temperature monitoring electrosurgical system 600 power output. Illustratively, full bridge RF drive 1005 and full bridge RF amplifier 1010 may be configured in a full-bridge configuration or a half-bridge configuration. In one or more embodiments, mono/bipolar configuration relays 1015 may be configured to direct a desired monopolar surgical power output to monopolar output transformer/filter 1020. Illustratively, mono/bipolar configuration relays 1015 may be configured to direct a desired bipolar surgical power output to bipolar output transformer/filter 1035. In one or more embodiments, bipolar output transformer/filter 1035 may be configured to prepare a temperature monitoring electrosurgical system 600 output power for bipolar HV relay 1065. Illustratively, bipolar HV relay 1065 may be configured to direct a temperature monitoring electrosurgical system 600 output power to temperature monitoring bipolar forceps assembly 200. In one or more embodiments, bipolar HV relay 1065 may be configured to modify an amount of voltage between conductor tips 110 via interface 1083. Illustratively, temperature monitoring system 900 may be configured to receive temperature data from first thermocouple 265 and second thermocouple 275 via interface 1083. In one or more embodiments, patient plate sense 1030 may be configured to receive information relating to grounding pad contact area via interface 1082. Illustratively, foot control HV relay may be configured to send signals to a foot pedal via interface 1081. In one or more embodiments, foot control HV relay may be configured to receive signals from a foot pedal via interface 1081. Illustratively, hand control sense 1055 may be configured to send signals to a remote controller via interface 1080. In one or more embodiments, hand control sense 1055 may be configured to receive signals from a remote controller via interface 1080. Illustratively, hand control HV relay 1050 may be configured to send signals to a remote controller via interface 1080. For example, hand control HV relay 1050 may be configured to receive signals from a remote controller via interface 1080. In one or more embodiments, voltage/current sense transformer 1045 may be configured to measure an output voltage and an output current. Illustratively, voltage/current sense transformer 1045 may be configured to measure an output voltage by measuring a voltage across a circuit element in parallel with an output load. In one or more embodiments, voltage/current sense transformer 1045 may be configured to measure an output current by measuring a total current into a node of the parallel circuit element and subtracting a curs rent through the parallel circuit element. Illustratively, analog scaling/ADC converters 1040 may be configured to convert a measured output voltage and a measured output current into signals that convey information about measured output voltage and measured output current to I/O controller 820.

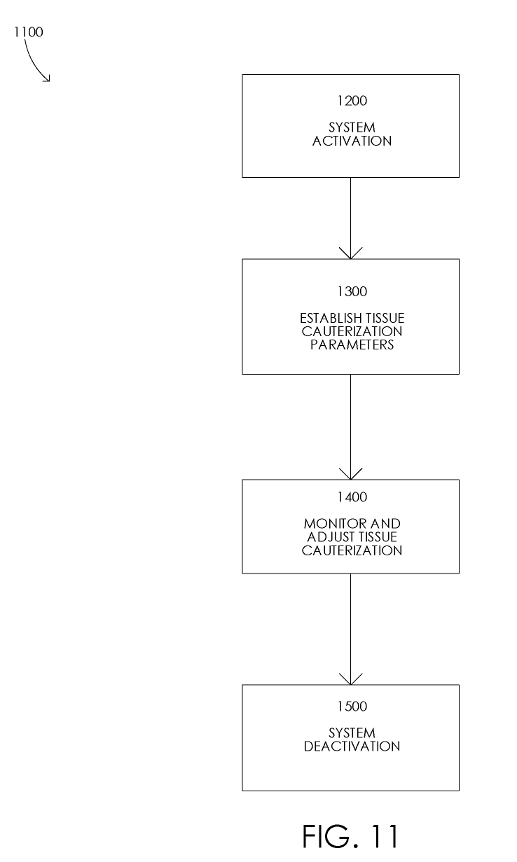

FIG. 11 is a flowchart illustrating a tissue cauterization 1100. Illustratively, a tissue cauterization 1100 may comprise system activation 1200, establishment of tissue cauterization parameters 1300, monitoring and adjustment of tissue cauterization 1400, and system deactivation 1500.

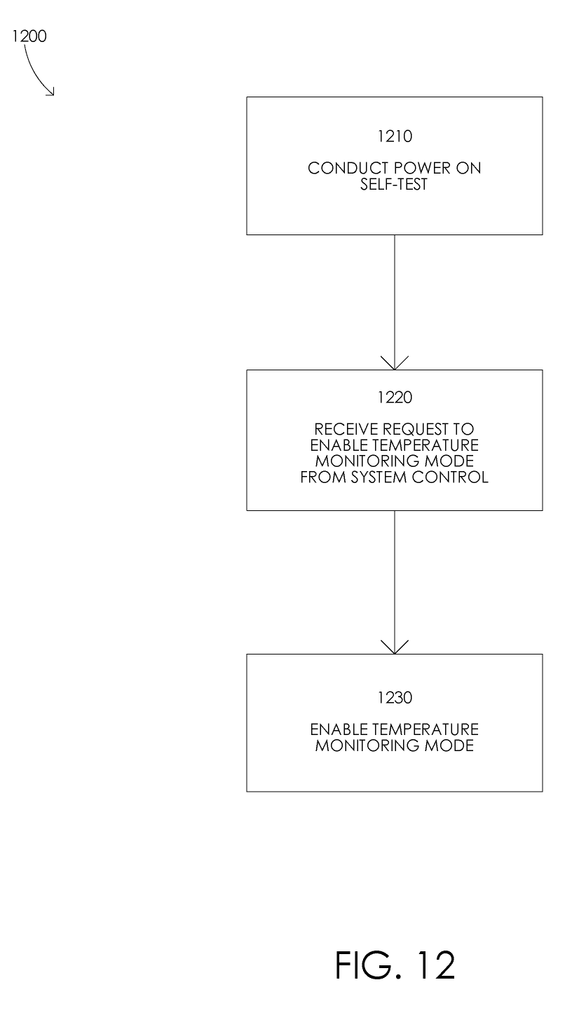

FIG. 12 is a flowchart illustrating a system activation 1200. Illustratively, system activation 1200 may comprise conducting power on self-test 1210, receiving request to enable temperature monitoring mode from system control 1220, and enabling temperature monitoring mode 1230. In one or more embodiments, conducting power on self-test 1210 may be configured to evaluate one or more components of temperature monitoring electrosurgical system 600, e.g., conducting power on self-test 1210 may be configured to ensure that all essential components of the electrosurgical generator are functioning as expected for performing a tissue cauterization 1100. Illustratively, a request to enable temperature monitoring mode may be initiated by a user, e.g., an assembled temperature monitoring bipolar forceps 300 may be packaged with an RFID card configured to enable temperature monitoring mode via user interface 610. In one or more embodiments, enabling temperature monitoring mode 1230 may be configured to activate temperature monitoring system 900, e.g., enabling temperature monitoring mode 1230 may be configured to cause first temperature sense 930 to evaluate an electrical condition of first thermocouple 265 and cause second temperature sense 931 to evaluate an electrical condition of second thermocouple 275.

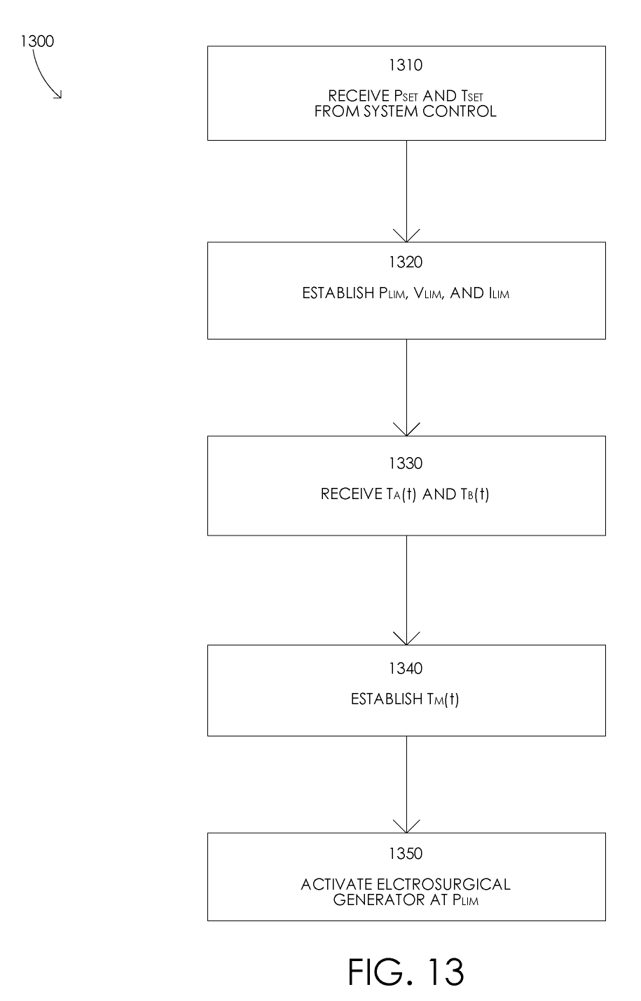

FIG. 13 is a flowchart illustrating an establishment of tissue cauterization parameters 1300. In one or more embodiments, an establishment of tissue cauterization parameters 1300 may comprise receiving a cauterization power setting (P_{SET}) and a cauterization temperature setting (T_{SET}) from system control 1310, establishing a cauterization power limit (P_{LIM}), a cauterization voltage limit (V_{LIM}), and a cauterization current limit (I_{LIM}) 1320, receiving a first thermocouple 265 temperature (T_{A}(t)) and a second thermocouple 275 temperature (T_{B}(t)) 1330, establishing a measured temperature (T_{M}(t)) 1340, and activating an electrosurgical generator at P_{LIM} 1350. Illustratively, receiving P_{SET} and T_{SET} from system control 1310 may comprise receiving P_{SET} and T_{SET} from a user input. In one or more embodiments, a user may specify P_{SET} and T_{SET} via user interface 610, e.g., enabling temperature monitoring mode 1230 may require a user to input P_{SET} and T_{SET}. Illustratively, P_{SET} may be configured to correspond to a user preferred cauterization power setting, e.g., P_{SET} may be set in watts, malis units, etc. In one or more embodiments, T_{SET} may be configured to correspond to a user preferred cauterization temperature setting, e.g., T_{SET} may be configured to correspond to an ideal cauterization temperature. Illustratively, system control 800 may receive P_{SET} and T_{SET} via user interface 610, e.g., system control 800 may communicate P_{SET} and T_{SET} to RF system 1000. In one or more embodiments, establishing P_{LIM}, V_{LIM}, and I_{LIM} 1320 may comprise measuring an impedance between conductor tips 110, referencing an impedance power curve, referencing a power setting power curve, and setting P_{LIM} to a power level based on P_{SET}. Illustratively, establishing P_{LIM}, V_{LIM}, and I_{LIM} 1320 may comprise referencing a peak voltage power curve and setting V_{LIM} to a voltage level based on P_{SET}. In one or more embodiments, establishing P_{LIM}, V_{LIM}, and I_{LIM} 1320 may comprise setting P_{LIM} equal to P_{SET}. Illustratively, receiving T_{A}(t) and T_{B}(t) 1330 may comprise receiving a first signal corresponding to a voltage between first wire conductor 266 and second wire conductor 268 and receiving a second signal corresponding to a voltage between third wire conductor 276 and fourth wire conductor 278. In one or more embodiments, receiving T_{A}(t) and T_{B}(t) 1330 may comprise converting the first signal into T_{A}(t) and converting the second signal into T_{B}(t). Illustratively, establishing T_{M}(t) 1340 may comprise preforming an operation on T_{A}(t) and T_{B}(t), e.g., establishing T_{M}(t) 1340 may comprise determining a maximum of T_{A}(t) and T_{B}(t) and setting T_{M}(t) equal to the maximum of T_{A}(t) and T_{B}(t). In one or more embodiments, establishing T_{M}(t) 1340 may comprise determining an average of T_{A}(t) and T_{B}(t) and setting T_{M}(t) equal to the average of T_{A}(t) and T_{B}(t). Illustratively, establishing T_{M}(t) 1340 may comprise determining a minimum of T_{A}(t) and T_{B}(t) and setting T_{M}(t) equal to the minimum of T_{A}(t) and T_{B}(t). In one or more embodiments, activating an electrosurgical generator at P_{LIM} 1350 may comprise delivering current to a surgical site. Illustratively, activating an electrosurgical generator at P_{LIM} 1350 may comprise establishing a cauterization voltage ((V(t)).

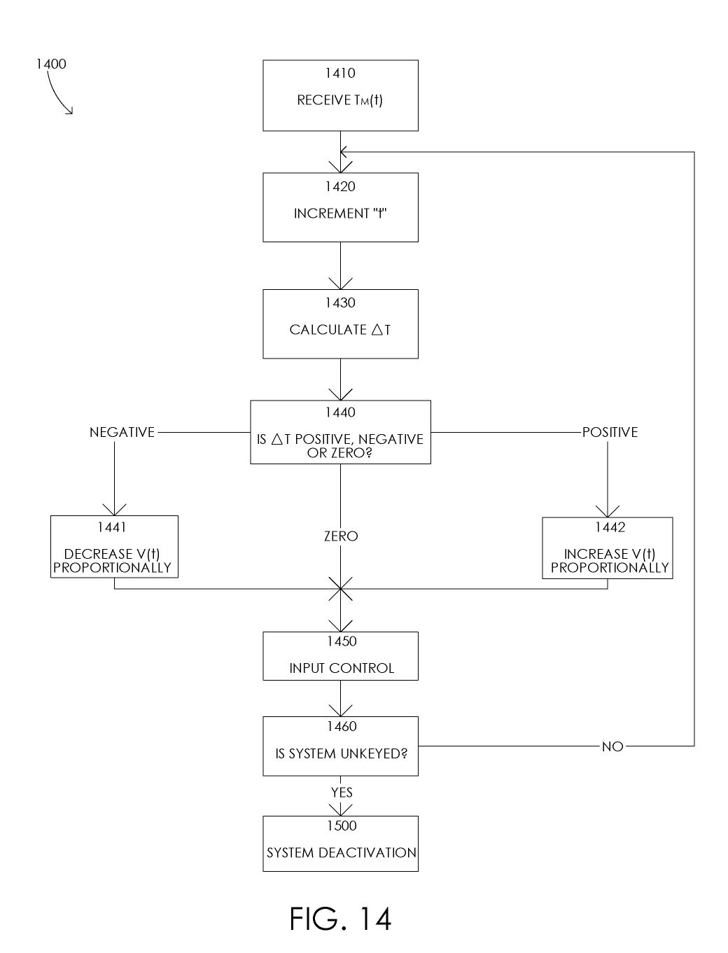

FIG. 14 is a flowchart illustrating a monitoring and adjustment of a tissue cauterization 1400. Illustratively, a monitoring and adjustment of a tissue cauterization 1400 may comprise receiving T_{M}(t) 1410, incrementing time (t) 1420, calculating a difference between T_{SET} and T_{M}(t) (ΔT) 1430, determining if ΔT is positive, negative, or zero 1440, inputting control 1450, determining if system is unkeyed 1460, and system deactivation 1500. In one or more embodiments, calculating ΔT 1430 may comprise subtracting T_{M}(t) from T_{SET}, e.g., if T_{SET} is less than T_{M}(t), then ΔT may be negative. Illustratively, if ΔT is negative, then system control 800 may be configured to decrease V(t), e.g., if ΔT is negative, then system control 800 may be configured to decrease V(t) proportionally to ΔT. In one or more embodiments, if a first ΔT is negative and a second ΔT is negative and if an absolute value of the first ΔT is greater than an absolute value of the second ΔT, then system control 800 may be configured to decrease V(t) by a first amount in response to the first ΔT and system control 800 may be configured to decrease V(t) by a second amount in response to the second ΔT wherein the first amount is greater than the second amount. Illustratively, if T_{SET} is greater than T_{M}(t), then ΔT may be positive. In one or more embodiments, if ΔT is positive, then system control 800 may be configured to increase V(t), e.g., if ΔT is positive, then system control 800 may be configured to increase V(t) proportionally to ΔT. Illustratively, if a first ΔT is positive and a second ΔT is positive and if an absolute value of the first ΔT is greater than an absolute value of the second ΔT, then system control 800 may be configured to increase V(t) by a first amount in response to the first ΔT and system control 800 may be configured to increase V(t) by a second amount in response to the second ΔT wherein the first amount is greater than the second amount. In one or more embodiments, inputting control 1450 may comprise adjusting an amount of power delivered to assembled temperature monitoring bipolar forceps 300, e.g., inputting control 1450 may comprise adjusting an amount of power delivered to assembled temperature monitoring bipolar forceps 300 based on V(t). Illustratively, decreasing V(t) may be configured to decrease T_{M}(t), e.g., if T_{SET} is less than T_{M}(t), then decreasing V(t) may be configured to modulate T_{M}(t) to match T_{SET}. In one or more embodiments, decreasing V(t) may be configured to decrease T_{A}(t). Illustratively, decreasing V(t) may be configured to decrease T_{B}(t). In one or more embodiments, decreasing V(t) may be configured to decrease T_{A}(t) and T_{B}(t). Illustratively, increasing V(t) may be configured to increase T_{M}(t), e.g., if T_{SET} is greater than T_{M}(t), then increasing V(t) may be configured to modulate T_{M}(t) to match T_{SET}. In one or more embodiments, increasing V(t) may be configured to increase T_{A}(t). Illustratively, increasing V(t) may be configured to increase T_{B}(t). In one or more embodiments, increasing V(t) may be configured to increase T_{A}(t) and T_{B}(t). Illustratively, determining if system is unkeyed 1460 may comprise system control 800 determining if a user is requesting power to assembled temperature monitoring bipolar forceps 300. In one or more embodiments, if system control 800 determines that a user is requesting power to assembled temperature monitoring bipolar forceps 300, then system control 800 may be configured to increment time (t). Illustratively, if system control 800 determines that a user is not requesting power to assembled temperature monitoring bipolar forceps 300, then system control 800 may be configured to initiate system deactivation 1500.

The foregoing description has been directed to particular embodiments of this invention. It will be apparent; however, that other variations and modifications may be made to the described embodiments, with the attainment of some or all of their advantages. Specifically, it should be noted that the principles of the present invention may be implemented in any system. Furthermore, while this description has been written in terms of an electrosurgical system, the teachings of the present invention are equally suitable to any systems where the functionality may be employed. Therefore, it is the object of the appended claims to cover all such variations and modifications as come within the true spirit and scope of the invention.

What is claimed is:

1. A system comprising:
a first forceps arm having a first forceps arm distal end and a first forceps arm proximal end;
a first conductor tip of the first forceps arm;
a first thermocouple electrically connected to a first temperature sense of a temperature monitoring system wherein the first thermocouple is electrically isolated from a system control of the system;
a second forceps arm having a second forceps arm distal end and a second forceps arm proximal end;
a second conductor tip of the second forceps arm;
a second thermocouple electrically connected to a second temperature sense of the temperature monitoring system wherein the second thermocouple is electrically isolated from the system control; and
a user interface of the system configured to receive a cauterization power setting and a cauterization temperature setting from a user wherein the system control is configured to establish a measured temperature from the first thermocouple and the second thermocouple and wherein the system control is configured to compare the measured temperature to the cauterization temperature setting.

2. The system of claim 1 wherein the system control is configured to increase a voltage between the first conductor tip and the second conductor tip if the measured temperature is less than the cauterization temperature setting.

3. The system of claim 1 wherein the system control is configured to decrease a voltage between the first conductor tip and the second conductor tip if the measured temperature is greater than the cauterization temperature setting.

4. The system of claim 1 wherein the system control is configured to establish one or more cauterization parameters.

5. The system of claim 4 wherein the system control is configured to establish a cauterization power limit.

6. The system of claim 5 wherein the cauterization power limit is equal to the cauterization power setting.

7. The system of claim 4 wherein the system control is configured to establish a cauterization voltage limit.

8. The system of claim 4 wherein the system control is configured to establish a cauterization current limit.

9. The system of claim 1 wherein the first thermocouple is electrically isolated from the system control by an opto-isolator.

10. The system of claim 1 further comprising:
an RF system of the system.

11. The system of claim 1 further comprising:
a power supply of the system.

12. The system of claim 1 further comprising:
an analog to digital converter of the temperature monitoring system.

13. The system of claim 12 wherein the analog to digital converter is electrically isolated from the system control.

14. The system of claim 1 wherein the first temperature sense is electrically isolated from the system control.

15. The system of claim 14 wherein the second temperature sense is electrically isolated from the system control.

16. A system comprising:
a first forceps arm having a first forceps arm distal end and a first forceps arm proximal end;
a first conductor tip of the first forceps arm;
a first thermocouple electrically connected to a first temperature sense of a temperature monitoring system wherein the first thermocouple is electrically isolated from a system control of the system;
a second forceps arm having a second forceps arm distal end and a second forceps arm proximal end;
a second conductor tip of the second forceps arm;
a second thermocouple electrically connected to a second temperature sense of the temperature monitoring system wherein the second thermocouple is electrically isolated from the system control; and
a user interface of the system configured to receive a cauterization power setting and a cauterization temperature setting from a user wherein the system control is configured to establish a measured temperature from the first thermocouple and the second thermocouple and wherein the system control is configured to compare the measured temperature to the cauterization temperature setting and wherein the system control is configured to increase a voltage between the first conductor tip and the second conductor tip if the measured temperature is less than the cauterization temperature setting and wherein the system control is configured to decrease the voltage between the first conductor tip and the second conductor tip if the measured temperature is greater than the cauterization temperature setting.

17. The system of claim 16 wherein the system control is configured to establish one or more cauterization parameters.

18. The system of claim 17 wherein the system control is configured to establish a cauterization power limit.

19. The system of claim 17 wherein the system control is configured to establish a cauterization voltage limit.

20. The system of claim 17 wherein the system control is configured to establish a cauterization current limit.
